(19) United States
(12) Patent Application Publication
(10) Pub. No.: US 2007/0274948 A1
(43) Pub. Date: Nov. 29, 2007

(54) Methods of Therapy for Chronic Lymphocytic Leukemia

(76) Inventors: Deborah Hurst, Emeryville, CA (US); Cornelia Quadt, Emeryville, CA (US); Maurice J. Wolin, Piedmont, CA (US); Sandra Milan, Emeryville, CA (US); Anders C. Osterborg, Stockholm, CA (US)

Correspondence Address: Davis Wright Tremaine;2600 Century Square, 1501 Fourth Avenue, Seatle, WA, 98101-1688, US

(21) Appl. No.: 10/566,410

(22) PCT Filed: Jun. 4, 2004
(86) PCT No.: PCT/US04/17921

§ 371 Date: May 29, 2007

Related U.S. Application Data
(60) Provisional application No. 60/491,371, filed on Jul. 30, 2003.

(51) Int. Cl. A61K 38/20; A61K 39/395; A61P 35/02

(52) U.S. Cl. 424/85.2; 424/133.1

(57) ABSTRACT

Methods for treating a human with chronic lymphocytic leukemia using a combination of an interleukin-2 and an anti-CD52 antibody are provided. These therapeutic agents are administered as two separate pharmaceutical compositions, one containing an IL-2, the other containing an anti-CD2 antibody, according to a dosing regiment. Administering of these two therapeutic agents together results in a positive therapeutic response that is improved with respect to that observed with anti-CD52 antibody alone.

### BACKGROUND OF THE INVENTION

Chronic lymphocytic leukemia (CLL) is the most common adult leukemia in the Western Hemisphere, and is generally fatal once the disease progresses. It mainly affects the elderly, and can have a long clinical course, up to 20 years after diagnosis. (Keating et al., (1992) Blood 99:3554-3561). The common therapies include alkylating agents such as chlorambucil (Binet (1992), Bailleres Clin. Hemat. 6:867-878), and purine analogs such as fludarabine (Grever et al., (1988) Nouv. Rev. Fr. Hematol. 30:457-459; Keating et al., (1998) Blood 92:1165-1171) or cladrabine (Saven (1996) Semin. Hematol. 33:28-33). Although fludarabine may be the most effective single agent, the CLL often becomes refractory to repeated courses of the same drug.

Interleukin-2 (IL-2) is a potent stimulator of natural killer (NK) and T-cell proliferation and function (Morgan et al. (1976) Science 193:1007-1011). This naturally occurring lymphokine has been shown to have anti-tumor activity against a variety of malignancies either alone or when combined with lymphokine-activated killer (LAK) cells or tumor-infiltrating lymphocytes (TIL) (see, for example, Rosenberg et al. (1987) N. Engl. J. Med. 316:889-897; Rosenberg (1988) Ann. Surg. 208:121-135; Topalian et al. (1988) J. Clin. Oncol. 6:839-853; Rosenberg et al. (1988) N. Engl. J. Med. 319:1676-1680; and Weber et al. (1992) J. Clin. Oncol. 10:33-40). The anti-tumor activity of IL-2 has best been described in patients with metastatic melanoma and renal cell carcinoma using Proleukin®IL-2, a commercially available IL-2 mutein formulation, aldesleukin.

More recently, the anti-CD52 antibody Alemtuzumab (Campath-1H) was approved and has been used to treat CLL. Alemtuzumab is a humanized monoclonal antibody directed against CD52, which is a cell surface protein expressed at high density on most normal and malignant B and T lymphocytes, but not on hemopoietic stem cells. However, anti-CD52 therapy can lead to depressed T-cell levels and associated opportunistic infections. New therapies are needed that will increase the safety and efficacy of anti-CD52 therapy for patients with advanced, chemotherapy refractory CLL.

### FIELD OF THE INVENTION

The present invention is directed to methods of therapy for chronic lymphocytic leukemia, more particularly to concurrent therapy with interleukin-2 and monoclonal antibodies targeting the CD52 B-cell surface antigen.

### BRIEF SUMMARY OF THE INVENTION

Methods for providing treatment to a human subject with chronic lymphocytic leukemia (CLL) using a combination of an interleukin-2 or a variant thereof (hereinafter collectively, “an IL-2”) and an anti-CD52 antibody or a fragment thereof (hereinafter collectively, “an anti-CD52 antibody”) are provided. These two therapeutic agents are administered as separate pharmaceutical compositions, one containing an IL-2, the other containing at least one anti-CD52 antibody, each according to a particular dosing regimen. The pharmaceutical composition comprising an anti-CD52 antibody is administered according to a weekly, twice-weekly, or thrice-weekly dosing schedule. The pharmaceutical composition comprising an IL-2 is administered according to a constant IL-2 dosing regimen, or is administered according to a two-level IL-2 dosing regimen. This two-level IL-2 dosing regimen comprises a first time period of IL-2 dosing, wherein a higher total weekly dose of an IL-2 is administered to the subject, followed by a second time period of IL-2 dosing, wherein a lower total weekly dose of an IL-2 is administered to the subject. Usually, the total weekly dose of an IL-2 during the second time period of IL-2 dosing is lower than the total weekly dose of an IL-2 administered during the first time period of IL-2 dosing. However, the total weekly dose of an IL-2 during the second time period may be higher than during the first period. Following one cycle of dosing of an IL-2 and an anti-CD52 antibody, a cycle of IL-2 alone may be administered for five consecutive days, followed by a period of weeks during which no IL-2 is administered. This cycle of IL-2 alone (five days of consecutive dosing followed by weeks of no IL-2 administration) can be repeated in order to restore T-cell counts.

The total weekly IL-2 dose to be administered during a constant IL-2 dosing regimen or during the first time period and/or during the second time period of an IL-2 dosing can be administered as a single dose. Alternatively, the total weekly dose administered can be partitioned into a series of equivalent doses that are administered according to a two-, three-, four-, five-, six- or seven-times-a-week dosing schedule. A preferred dosing schedule is three times weekly.

Although it is preferable to give one cycle of approximately 12 weeks with an anti-CD52 antibody, in some embodiments, two or multiple cycles of therapy with an anti-CD52 antibody in combination with IL-2 dosing are administered to a subject in need of treatment for CLL, wherein each cycle comprises administering an anti-CD52 antibody in combination with the constant IL-2 dosing regimen or the two-level IL-2 dosing regimen. The need for administering these multiple cycles is assessed, for example, by monitoring natural-killer (NK) cell counts and T cell counts in subjects undergoing treatment with the methods of the invention. The methods also provide for an interruption in the two-level dosing regimen of IL-2, where the subject is given a time period off of IL-2 administration, or a time period off of IL-2 and anti-CD52 antibody administration, between the first and second time periods of the two-level IL-2 dosing regimen.

The methods of the invention provide a means for reducing side effects of anti-CD52 antibody administration, thereby improving the safety of treatment with this therapeutic agent. The methods may also provide a means for stimulating NK cells/ADCC, the most important effector function of anti-CD52 antibody therapy.

The invention also provides for the use of an IL-2 or an anti-CD52 antibody in the manufacture of a medicament for use in treatment of CLL. In some embodiments, the anti-CD52 antibody is Alemtuzumab.

Also provided is a kit comprising an anti-CD52 antibody, and/or an IL-2, and instructions for delivering the anti-CD52 antibody and the IL-2 to an individual suffering from CLL wherein the instructions provide for one cycle of concurrent therapy with the anti-CD52 antibody and the IL-2. In some embodiments of the kit, the anti-CD52 antibody is Alemtuzumab.

The invention provides a product containing an anti-CD52 antibody and an IL-2 as a combined preparation for simultaneous, separate, or sequential use in CLL therapy. Although the components (an anti-CD52 antibody and an IL-2) do not enter into a direct interaction with each other in the product itself, they are intended for combined therapy for CLL. The two components (an anti-CD52 antibody and an IL-2) can be applied simultaneously, separately, or at intervals as specified herein, to one and the same human in need thereof.

In certain embodiments of the product, an anti-CD52 antibody is an immunologically active anti-CD52 antibody. In further embodiments of the product, an anti-CD52 antibody is Alemtuzumab or fragment thereof. In yet further embodiments of the product, an anti-CD52 antibody is a human anti-CD52 antibody, a humanized anti-CD52 antibody, or a chimeric anti-CD52 antibody.

In embodiments of the product an IL-2 is recombinantly produced IL-2 having an amino acid sequence for human IL-2 or a variant thereof having at least 70% sequence identity to the amino acid sequence for human IL-2. In further embodiments of the product, the variant of human IL-2 is des-alanyl-1, serine 125 human interleukin-2. The product can be formulated according to any and all doses and cycles as disclosed herein.

The invention provides a method of treating chronic lymphocytic leukemia in a human subject, the method comprising administering to the subject at least one cycle of concurrent therapy with an anti-CD52 antibody and an interleukin-2 (IL-2).

In some aspects of this method, the cycle comprises administering a therapeutically effective dose of an anti-CD52 antibody in combination with administration of a two-level dosing regimen of IL-2. In certain embodiments, the anti-CD52 antibody is administered according to a weekly, twice weekly, or three times weekly dosing schedule.

In further aspects of the method, the two-level dosing regimen of an IL-2 comprises a first time period, wherein a higher total weekly dose of an IL-2 is administered to the subject, followed by a second time period, wherein a lower total weekly dose of IL-2 is administered to the subject.

In other aspects of the method, the two-level dosing regimen of an IL-2 comprises a first time period, wherein a lower total weekly dose of an IL-2 is administered to the subject, followed by a second time period, wherein a higher total weekly dose of an IL-2 is administered to the subject.

In some embodiments, the first dose of an IL-2 is administered to the subject concurrently with a first dose of an anti-CD52 antibody. In further embodiments of the two-level dosing regimen of an IL-2, a first dose of an IL-2 is administered to the subject one week after a first dose of an anti-CD52 antibody is administered to the subject. In certain embodiments, the anti-CD52 antibody is dosed weekly for 8 weeks to 12 weeks, and the therapeutically effective dose of the anti-CD52 antibody is in the range from about 10 mg to about 30 mg. The two-level dosing regimen of an IL-2 may have a combined duration of 10 weeks to 20 weeks, and wherein IL-2 dosing continues for about 2 weeks to about 6 weeks longer than anti-CD52 antibody administration. The first time period of the two-level dosing regimen of an IL-2 may have a duration that is one-half of the combined duration of 10 weeks to 20 weeks.

The invention provides a method of treating chronic lymphocytic leukemia in a human subject, the method comprising administering to the subject at least one cycle of concurrent therapy with an anti-CD52 antibody and an interleukin-2 (IL-2), wherein in some embodiments, a higher total weekly dose of an IL-2 is administered as a single dose or is partitioned into a first series of equivalent doses that are administered according to a two-, three-, four-, five-, six- or seven-times-a-week dosing schedule, and a lower total weekly dose of an IL-2 is administered as a single dose or is partitioned into a second series of equivalent doses that are administered according to a two-, three-, four-, five-, six- or seven-times-a-week dosing schedule. In some embodiments, the IL-2 is administered by a subcutaneous route.

In further embodiments of this method, the higher total weekly dose of an IL-2 is in an amount equivalent to a total weekly dose of aldesleukin in a range from 1834 μg to 2565 μg and the lower total weekly dose of an IL-2 is an amount equivalent to a total weekly dose of aldesleukin, wherein the higher and lower total weekly doses of the IL-2 provide at least 50% of the NK stimulatory activity of the total weekly doses of aldesleukin. In some embodiments, the higher total weekly dose of an IL-2 is administered as a single dose or is partitioned into a first series of equivalent doses that are administered according to a two-, three-, four-, five-, six- or seven-times-a-week dosing schedule, and the lower total weekly dose of an IL-2 is administered as a single dose or is partitioned into a second series of equivalent doses that are administered according to a two-, three-, four-, five-, six- or seven-times-a-week dosing schedule. The therapeutically effective dose of the anti-CD52 antibody may be in the range from about 10 mg to about 30 mg.

The invention provides a method of treating chronic lymphocytic leukemia in a human subject, comprising administering to the subject at least one cycle of concurrent therapy with an anti-CD52 antibody and an interleukin-2 (IL-2), wherein the IL-2 is provided in a pharmaceutical composition selected from the group consisting of a monomeric IL-2 pharmaceutical composition, a multimeric IL-2 composition, a stabilized lyophilized IL-2 pharmaceutical composition, and a stabilized spray-dried IL-2 pharmaceutical composition. The IL-2 may be recombinantly produced IL-2 having an amino acid sequence for human IL-2 or a variant thereof having at least 70% sequence identity to the amino acid sequence for human IL-2. In some embodiments, the variant is des-alanyl-1, serine 125 human interleukin-2.

The invention provides a method of treating chronic lymphocytic leukemia in a human subject, comprising administering to the subject at least one cycle of concurrent therapy with an anti-CD52 antibody and an interleukin-2 (IL-2), wherein the anti-CD52 antibody is an immunologically active anti-CD52 antibody. The anti-CD52 antibody may be Alemtuzumab or fragment thereof. The anti-CD52 antibody may be a human anti-CD52 antibody, a humanized anti-CD52 antibody, or a chimeric anti-CD52 antibody.

The invention provides a method of treating chronic lymphocytic leukemia in a human subject, comprising administering to the subject at least one cycle of concurrent therapy with an anti-CD52 antibody and an interleukin-2 (IL-2), wherein one or more subsequent cycles of concurrent therapy with an IL-2 and an anti-CD52 antibody may be initiated about 1 month to about 6 months following completion of a first cycle or completion of any subsequent cycles of concurrent therapy with an IL-2 and an anti-CD52 antibody. In these embodiments, T-cell counts may be monitored in the subject to determine when each of the cycles is initiated, with the cycles being initiated when the T-cell count is less than 80% of the T-cell count at the conclusion of any previous cycle of concurrent therapy with an IL-2 and an anti-CD52 antibody.

In embodiments wherein the cycle comprises administering a therapeutically effective dose of an anti-CD52 antibody in combination with administration of a two-level dosing regimen of IL-2, the invention provides for an interruption in the two-level dosing regimen of an IL-2, the interruption comprising a time period off of IL-2 administration between the first time period and the second time period of the two-level dosing regimen of IL-2. In some embodiments, the interruption further comprises a time period off of anti-CD52 antibody administration. In such embodiments, T-cell counts are monitored in the human to determine when the second time period of the two-level IL-2 dosing regimen is initiated, the second time period being initiated when T-cell count is less than 80% of the T-cell count at the conclusion of the first time period of the two-level IL-2 dosing regimen. The interruption may have a duration of about 1 week to about 4 weeks.

The invention still further provides a method of treating chronic lymphocytic leukemia in a human subject, the method comprising administering to the subject a cycle of concurrent therapy with an anti-CD52 antibody and an interleukin-2 (IL-2) comprising the steps of: (a) administering 3 mg, 10 mg, and 30 mg of anti-CD52 antibody, respectively, on days 1,3, and 5, subcutaneously; (b) administering an IL-2 at a dose of at least 10 MIU subcutaneously three times weekly for 15 weeks; and (c) continuing to administer the anti-CD52 antibody three times weekly at 30 mg per dose for 11 weeks and the IL-2 at a dose of at least 10 MIU subcutaneously on the same days as the anti-CD52 antibody and for 4 weeks following cessation of the CD52 antibody therapy, wherein the anti-CD52 antibody is Alemtuzumab and the IL-2 is Proleukin. In some embodiments, the dose of IL-2 is 14 MIU.

The invention also provides a method of treating chronic lymphocytic leukemia in a human subject, the method comprising administering to the subject at least one cycle of concurrent therapy with an anti-CD52 antibody and an interleukin-2 (IL-2), wherein the cycle comprises administering a therapeutically effective dose of an anti-CD52 antibody according to a weekly, twice-weekly, or thrice-weekly dosing schedule in combination with administration of a constant IL-2 dosing regimen, the constant IL-2 dosing regimen comprising administering a total weekly dose of an IL-2 to the subject. In some embodiments, a first dose of an IL-2 is administered to the subject concurrently with a first dose of an anti-CD52 antibody. The first dose of an IL-2 may be administered to the subject one week after a first dose of an anti-CD52 antibody is administered to the subject, and the anti-CD52 antibody may be dosed weekly for 8 weeks to 12 weeks. The therapeutically effective dose of the anti-CD52 antibody may be in the range from about 10 mg to about 30 mg. The anti-CD52 antibody may be dosed subcutaneously thrice weekly.

In further embodiments of this constant IL-2 dosing regimen, the IL-2 dosing regimen has a combined duration of 10 weeks to 20 weeks, wherein IL-2 dosing continues for about 2 weeks to about 6 weeks longer than anti-CD52 antibody administration. The total weekly dose of IL-2 may be administered as a single dose or may be partitioned into a first series of equivalent doses that are administered according to a two-, three-, four-, five-, six- or seven-times-a-week dosing schedule. The IL-2 may be administered by a subcutaneous route.

In the constant dosing regimen for IL-2, the total weekly dose of an IL-2 is in an amount equivalent to a total weekly dose of aldesleukin in a range from 1100 μg to 2565 μg, wherein the total weekly dose of an IL-2 is an amount that provides at least 50% of the NK stimulatory activity of the total weekly dose of aldesleukin. The total weekly dose of an IL-2 may be administered as a single dose or may be partitioned into a first series of equivalent doses that are administered according to a two-, three-, four-, five-, six- or seven-times-a-week dosing schedule. The therapeutically effective dose of an anti-CD52 antibody may be about 10 mg to about 30 mg, and the therapeutically effective dose of an anti-CD52 antibody may be administered subcutaneously three times weekly. The total weekly dose of an IL-2 may be administered thrice-weekly, and the total weekly dose of an IL-2 may be 1834 μg. The total weekly dose of an IL-2 may be administered thrice-weekly, and the total weekly dose of an IL-2 may be 1100 μg. The therapeutically effective dose of the anti-CD52 antibody may be in the range from about 10 mg to about 30 mg.

In certain embodiments, the IL-2 may be provided in a pharmaceutical composition selected from the group consisting of a monomeric IL-2 pharmaceutical composition, a multimeric IL-2 composition, a stabilized lyophilized IL-2 pharmaceutical composition, and a stabilized spray-dried IL-2 pharmaceutical composition. In further embodiments, the IL-2 may be recombinantly produced IL-2 having an amino acid sequence for human IL-2 or a variant thereof having at least 70% sequence identity to the amino acid sequence for human IL-2. In certain embodiments, the variant thereof may be des-alanyl-1, serine 125 human interleukin-2. In still further embodiments, the anti-CD52 antibody is an immunologically active anti-CD52 antibody, and the anti-CD52 antibody may be Alemtuzumab or fragment thereof. The anti-CD52 antibody may be a human anti-CD52 antibody, a humanized anti-CD52 antibody, or a chimeric anti-CD52 antibody.

In further embodiments of a method of treating CLL in a human subject, one or more subsequent cycles of concurrent therapy with IL-2 and anti-CD52 antibody is initiated about 1 month to about 6 months following completion of a first cycle or completion of any subsequent cycles of concurrent therapy with IL-2 and anti-CD52 antibody. In such embodiments, the T-cell counts are monitored in the subject to determine when each of the cycles is initiated, the cycles being initiated when the T-cell count is less than 80% of the T-cell count at the conclusion of any previous cycle of concurrent therapy with an IL-2 and an anti-CD52 antibody. In further embodiments, the total weekly dose of an IL-2 is in an amount that provides at least 50% of the NK stimulatory activity of a total weekly dose of Aldesleukin administered in a range of from about 1100 μg to about 1834 μg, and the NK stimulatory activity may be measured in K562 cells by ⁵¹Cr release assay.

In still further embodiments of a method of treating CLL in a human subject, the total weekly dose of an IL-2 is an amount that provides at least 60% of the NK stimulatory activity of a total weekly dose of aldesleukin administered in a range of from about 1100 μg to about 1834 μg; or at least 70% of the NK stimulatory activity of a total weekly dose of aldesleukin administered in a range of from about 1100 μg to about 1834 μg; or at least 80% of the NK stimulatory activity of a total weekly dose of aldesleukin administered in a range of from about 1100 μg to about 1834 μg; or at least 90% of the NK stimulatory activity of a total weekly dose of aldesleukin administered in a range of from about 1100 μg to about 1834 μg. The total weekly dose of an IL-2 may be administered as a single dose or may be partitioned into a first series of equivalent doses that are administered according to a two-, three-, four-, five-, six- or seven-times-a-week dosing schedule. In these embodiments, the therapeutically effective dose of an anti-CD52 antibody is about 10 mg to about 30 mg, and the therapeutically effective dose of an anti-CD52 antibody is administered subcutaneously thrice-weekly.

The invention still further provides for the use of an IL-2 in the manufacture of a medicament for use in treatment of CLL in an individual concurrently treated with an anti-CD52 antibody. In certain embodiments, the anti-CD52 antibody is Alemtuzumab.

The invention also provides a kit comprising an anti-CD52 antibody and instructions for delivering the anti-CD52 antibody to an individual suffering from CLL, concurrently with delivery of an IL-2, wherein the instructions provide for one cycle of concurrent therapy with the anti-CD52 antibody and the IL-2. In certain embodiments, the anti-CD52 antibody is Alemtuzumab.

The invention yet further provides a kit comprising an IL-2 and instructions for delivering the IL-2 to an individual suffering from CLL, concurrently with delivery of an anti-CD52 antibody, wherein the instructions provide for one cycle of concurrent therapy with the IL-2 and the anti-CD52 antibody. In certain embodiments, the IL-2 is aldesleukin.

The invention also provides a product containing an anti-CD52 antibody and an IL-2 as a combined preparation for simultaneous, separate, or sequential use in CLL therapy. In certain embodiments, the anti-CD52 antibody may be an immunologically active anti-CD52 antibody; the anti-CD52 antibody may be Alemtuzumab or fragment thereof; or the anti-CD52 antibody may be a human anti-CD52 antibody, a humanized anti-CD52 antibody, or a chimeric anti-CD52 antibody. In some embodiments of this product, the IL-2 is recombinantly produced IL-2 having an amino acid sequence for human IL-2 or a variant thereof having at least 70% sequence identity to the amino acid sequence for human IL-2. The variant thereof may be des-alanyl-1, serine 125 human interleukin-2.

### BRIEF DESCRIPTION OF THE DRAWING

FIG. 1. FIG. 1 is a chart depicting a preferred study design for IL-2 and Alemtuzumab, with the times for initial testing and ongoing testing during the course of treatment. IC refers to informed consent.

### DETAILED DESCRIPTION OF THE INVENTION

Alemtuzumab, also known as Campath®/MabCampath™, is approved for the treatment of chronic lymphocytic leukemia in patients who have failed treatment with fludarabine. The monoclonal antibody, directed against the CD52 cell surface antigen, has efficacy with response rates of 33-43% in fludarabine-refractory CLL patients, and a median duration of response of about 9 months. Side effects are significant and include immunosuppression, in particular depressed T cells, which leads to an increased risk of opportunistic infections including Candida, CMV, Aspergillosis, Pneumocystis Carinii, and Herpes zoster. The basis of the present invention is that by administering an IL-2 to patients receiving anti-CD52 antibody treatment, some or all of these T-cell effects will be abrogated, and at a minimum T-cell counts would be preserved. The rationale for administering an IL-2 is at least two-fold: to reduce the side-effects of a concomitant anti-CD52 antibody treatment; and to promote restoration of T-cell levels that may be depleted by an anti-CD52 antibody treatment. In view of these goals, it is most appropriate to administer an IL-2 at a time when an anti-CD52 antibody is having or has had an effect on T-cells. The advantage of this approach is that anti-CD52 antibody therapy could be continued in more patients than otherwise and with a reduced risk of serious infectious complications. IL-2 itself has anti-tumor activity. In a further advantage, as IL-2 also stimulates NK-cells, the antitumor effects of anti-CD52 antibody may be enhanced by concomitant IL-2 administration.

CD52 and antibodies directed against CD52 have been the subject of recent study in relation to lymphoid malignancies and immune system-related disorders, including rheumatoid arthritis. These studies are described in, for example, Taylor, V. C. et al., Biochem. J. 322:919-925 (1997); Franchiolla, N. S. et al., Brit. Jour. Haematol. 112:969-971, 2001; Clarke, E. et al., Bone Marrow Transplantation 20:599-605, 1997; Schnell, R. et al., Biodrugs 8:216-234, 1997; Condiotti, R. et al., Bone Marrow Transplantation 18:713-720, 1996; Pruzanski, W. et al., Jour. Rheumatol. 22:1816-1819, 1995; Lundin J et al., Blood 2003, June 1 (in press), Lundin, J. et al., Blood 100:768-773, 2002; Keating, M. J. et al., Blood 99:355-43561, 2002; Osterborg, A. et al., J Clin Oncol 15:1567-1574, 1997, and Lundin J et al., J Clin Oncol 16:3257-3263, 1998.

The present invention therefore relates to methods of treating a human subject with leukemia, more particularly chronic lymphocytic leukemia (CLL). The methods are also suitable for treating B-cell non-Hodgkin's lymphoma. The methods comprise combination therapy with an anti-CD52 antibody or a fragment thereof (hereinafter collectively, “an anti-CD52 antibody” as defined below) and an interleukin-2 (hereinafter collectively, “an IL-2” as defined below). Combination therapy with these two therapeutic agents provides for increased safety and potentially also increased anti-tumor activity. By increased safety is intended less reduced numbers and/or improved function of normal T-cells, which may in turn lead to a reduced incidence of severe infectious complications otherwise induced by anti-CD52 therapy. By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Subjects undergoing therapy with a combination of an IL-2 and an anti-CD52 antibody in accordance with the methods of the present invention are expected to experience a physiological response that is beneficial with respect to the risk of severe complications induced by anti-CD52 treatment of leukemia, more particularly chronic lymphocytic leukemia.

The therapeutic methods of the invention are directed to treatment of any chronic lymphocytic leukemia whose abnormal B-cell type expresses the CD52 surface antigen. By “CD52 surface antigen” is intended a 21-28 kD cell surface glycoprotein that is expressed on the surface of normal and malignant B and T lymphocytes, NK cells, monocytes, macrophages, and tissues of the male reproductive system.

In one embodiment, an anti-CD52 antibody is the Campath-1H antibody (Alemtuzumab). This antibody is an IgG1 kappa with human variable framework and constant regions, and complementarity-determining regions from a murine (rat) monoclonal antibody (Campath-1G). The Campath-1H antibody has an approximate molecular weight of 150 kD. The proposed mode of action of the antibody is ADCC of leukemic cells following cell-surface binding of the CD52 surface antigen.

The pharmacokinetic profile of Alemtuzumab was studied in a multicenter rising-dose trial in non-Hodgkin's lymphoma and CLL. Campath® was administered once weekly for a maximum of 12 weeks. Following intravenous infusions over a range of doses, the maximum serum concentration (Cₘₐₓ) and the area under the curve (AUC) showed relative dose proportionality. The overall average half-life over the dosing interval was about 12 days. The pharmacokinetic profile of Campath® administered as a 30 mg intravenous infusion three times per week was evaluated in CLL patients. Peak and trough levels of Campath® rose during the first few weeks of I.V. treatment, and appeared to approach a steady state by approximately week 6, although there was some inter-patient variability. The rise in serum Campath® concentration corresponded with the elimination of malignant lymphocytosis.

Definitions: Anti-CD52 antibody. As used herein, the term “an anti-CD52 antibody” encompasses any one or more antibody that specifically recognizes the CD52 B-cell surface antigen, including polyclonal anti-CD52 antibodies, monoclonal anti-CD52 antibodies, human anti-CD52 antibodies, humanized anti-CD52 antibodies, chimeric anti-CD52 antibodies, xenogeneic anti-CD52 antibodies, and fragments of these anti-CD52 antibodies that specifically recognize the CD52 B-cell surface antigen. Preferably the antibody is monoclonal in nature. By “monoclonal antibody” is intended an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site, i.e., the CD52 B-cell surface antigen in the present invention. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J. Mol. Biol. 222:581-597, for example.

Anti-CD52 antibodies of murine origin are also suitable for use in the methods of the present invention. The term “an anti-CD52 antibody” as used herein encompasses humanized anti-CD52 antibodies. By “humanized antibodies” is intended antibodies that are most preferably derived using recombinant deoxyribonucleic acid techniques and which comprise both human (including immunologically “related” species, e.g., chimpanzee) and non-human components. Thus, the constant region of the chimeric antibody is most preferably substantially identical to the constant region of a natural human antibody; the CDR-regions of the humanized antibody is most preferably derived from a non-human source and has the desired antigenic specificity to the CD52 cell surface antigen. The non-human source can be any vertebrate source that can be used to generate antibodies to a human CD52 cell surface antigen or material comprising a human CD52 cell surface antigen. Such non-human sources include, but are not limited to, rodents (e.g., rabbit, rat, mouse, etc.; see, for example, U.S. Pat. No. 4,816,567) and non-human primates (e.g., Old World Monkey, Ape, etc.; see, for example, U.S. Pat. Nos. 5,750,105 and 5,756,096). Most preferably, the non-human component (CDR regions) is derived from a murine source. As used herein, the phrase “immunologically active” when used in reference to humanized anti-CD52 antibodies means a humanized antibody that binds human CD52, mediates complement dependent lysis (“CDC”) of human cell lines expressing CD52, and lyses human target cells through antibody dependent cellular cytotoxicity (“ADCC”). Examples of humanized anti-CD52 antibodies include, but are not limited to, Alemtuzumab, available commercially under the name Campath® (Berlex Laboratories, Richmond, Calif.), and described in U.S. Pat. Nos. 5,545,403; 5,545,405; 5,654,403; and 5,846,534.

Chimeric anti-CD52 antibodies are also encompassed by the term “an anti-CD52 antibody” as used herein. By “chimeric” is intended forms of anti-CD52 antibodies that for the most part, are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. See, for example, U.S. Pat. Nos. 5,225,539; 5,585,089; 5,693,761; 5,693,762; 5,859,205. In some instances, framework residues of the human immunoglobulin are replaced by corresponding non-human residues (see, for example, U.S. Pat. Nos. 5,585,089; 5,693,761; 5,693,762). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al. (1986) Nature 331:522-525; Riechmann et al. (1988) Nature 332:323-329; and Presta (1992) Curr. Op. Struct. Biol. 2:593-596.

Other methods of humanizing antibodies are disclosed in, for example, U.S. Pat. Nos. 5,595,721 and 6,054,561.

A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J Immunol. 138:4534-4538; and Brown et al. (1987) Cancer Res. 47:3577-3583), rodent CDRs grafted into a human supporting framework repair (FR) prior to fusion with an appropriate human antibody constant domain (Reichmann et al. (1988) Nature 332:323-327; Verhoeyen et al. (1988) Science 239:1534-1536; and Jones et al. (1986) Nature 321:522-525), and rodent CDRs supported by recombinantly veneered rodent FRs (European Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients.

As used herein, the terms “veneered FRs” and “recombinantly veneered FRs” refer to the selective replacement of FR residues from, e.g., a rodent heavy or light chain V region, with human FR residues in order to provide a xenogeneic molecule comprising an antigen-binding site which retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface. Davies et al. (1990) Ann. Rev. Biochem. 59:439-473. Thus, antigen binding specificity can be preserved in a humanized antibody only wherein the CDR structures, their interaction with each other, and their interaction with the rest of the V region domains are carefully maintained. By using veneering techniques, exterior (e.g., solvent-accessible) FR residues which are readily encountered by the immune system are selectively replaced with human residues to provide a hybrid molecule that comprises either a weakly immunogenic, or substantially non-immunogenic, veneered surface.

The process of veneering makes use of the available sequence data for human antibody variable domains compiled by Kabat et al., in Sequences of Proteins of Immunological Interest, 4th ed. (U.S. Dept. of Health and Human Services, U.S. Government Printing Office, 1987), updates to the Kabat database, and other accessible U.S. and foreign databases (both nucleic acid and protein). Solvent accessibilities of V region amino acids can be deduced from the known three-dimensional structure for human and murine antibody fragments. There are two general steps in veneering a murine antigen-binding site. Initially, the FRs of the variable domains of an antibody molecule of interest are compared with corresponding FR sequences of human variable domains obtained from the above-identified sources. The most homologous human V regions are then compared residue by residue to corresponding murine amino acids. The residues in the murine FR which differ from the human counterpart are replaced by the residues present in the human moiety using recombinant techniques well known in the art. Residue switching is only carried out with moieties which are at least partially exposed (solvent accessible), and care is exercised in the replacement of amino acid residues which may have a significant effect on the tertiary structure of the V region domains, such as proline, glycine and charged amino acids.

In this manner, the resultant “veneered” murine antigen-binding sites are thus designed to retain the murine CDR residues, the residues substantially adjacent to the CDRs, the residues identified as buried or mostly buried (solvent inaccessible), the residues believed to participate in non-covalent (e.g., electrostatic and hydrophobic) contacts between heavy and light chain domains, and the residues from conserved structural regions of the FRs which are believed to influence the “canonical” tertiary structures of the CDR loops. These design criteria are then used to prepare recombinant nucleotide sequences which combine the CDRs of both the heavy and light chain of a murine antigen-binding site into human-appearing FRs that can be used to transfect mammalian cells for the expression of recombinant human antibodies which exhibit the antigen specificity of the murine antibody molecule.

Also encompassed by the term “an anti-CD52 antibody” are xenogeneic or modified anti-CD52 antibodies produced in a non-human mammalian host, more particularly a transgenic mouse, characterized by inactivated endogenous immunoglobulin (Ig) loci. In such transgenic animals, competent endogenous genes for the expression of light and heavy subunits of host immunoglobulins are rendered non-functional and substituted with the analogous human immunoglobulin loci. These transgenic animals produce human antibodies in the substantial absence of light or heavy host immunoglobulin subunits. See, for example, U.S. Pat. No. 5,939,598.

Fragments of anti-CD52 antibodies are suitable for use in the methods of the invention so long as they retain the desired affinity of a full-length antibody. Thus, a fragment of an anti-CD52 antibody will retain the ability to bind to the CD52 surface antigen. Fragments of an antibody comprise a portion of a full-length antibody, generally the antigen binding or variable region thereof. Examples of antibody fragments include, but are not limited to, Fab, Fab′, F(ab′)₂, and Fv fragments and single-chain antibody molecules. By “single-chain Fv” or “sFv”)-antibody fragments is intended fragments comprising the V_{H} and V_{L} domains of an antibody, wherein these domains are present in a single polypeptide chain. See, for example, U.S. Pat. Nos. 4,946,778; 5,260,203; 5,455,030; 5,856,456. Generally, the Fv polypeptide further comprises a polypeptide linker between the V_{H} and V_{L} domains that enables the sFv to form the desired structure for antigen binding. For a review of sFv see Pluckthun (1994) in The Pharmacology of Monoclonal Antibodies, Vol. 113, ed. Rosenburg and Moore (Springer-Verlag, New York), pp. 269-315.

Antibodies or antibody fragments can be isolated from antibody phage libraries generated using the techniques described in McCafferty et al. (1990) Nature 348:552-554 (1990). Clackson et al. (1991) Nature 352:624-628 and Marks et al. (1991) J. Mol. Biol. 222:581-597 describe the isolation of murine and human antibodies, respectively, using phage libraries. Subsequent publications describe the production of high affinity (nM range) human antibodies by chain shuffling (Marks et al. (1992) Bio/Technology 10:779-783), as well as combinatorial infection and in vivo recombination as a strategy for constructing very large phage libraries (Waterhouse et al. (1993) Nucleic. Acids Res. 21:2265-2266). Thus, these techniques are viable alternatives to traditional monoclonal antibody hybridoma techniques for isolation of monoclonal antibodies.

A humanized antibody has one or more amino acid residues introduced into it from a source that is non-human. These non-human amino acid residues are often referred to as “donor” residues, which are typically taken from a “donor” variable domain. Humanization can be essentially performed following the method of Winter and co-workers (Jones et al. (1986) Nature 321.522-525; Riechmann et al. (1988) Nature 332:323-327; Verhoeyen et al. (1988) Science 239: 1534-1536), by substituting rodent CDRs or CDR sequences for the corresponding sequences of a human antibody. See, for example, U.S. Pat. Nos. 5,225,539; 5,585,089; 5,693,761; 5,693,762; 5,859,205. Accordingly, such “humanized” antibodies may include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205. See also U.S. Pat. No. 6,180,370, and International Publication No. WO 01/27160, where humanized antibodies and techniques for producing humanized antibodies having improved affinity for a predetermined antigen are disclosed.

Various techniques have been developed for the production of antibody fragments. Traditionally, these fragments were derived via proteolytic digestion of intact antibodies (see, e.g., Morimoto et al. (1992) Journal of Biochemical and Biophysical Methods 24:107-117 (1992) and Brennan et al. (1985) Science 229:81). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al. (1992) Bio/Technology 10:163-167). According to another approach, F(ab′)₂ fragments can be isolated directly from recombinant host cell culture. Other techniques for the production of antibody fragments will be apparent to the skilled practitioner.

Further, any of the previously described anti-CD52 antibodies may be conjugated prior to use in the methods of the present invention. Such conjugated antibodies are available in the art. Thus, an anti-CD52 antibody may be labeled using an indirect labeling or indirect labeling approach. By “indirect labeling” or “indirect labeling approach” is intended that a chelating agent is covalently attached to an antibody and at least one radionuclide is inserted into the chelating agent. See, for example, the chelating agents and radionuclides described in Srivagtava and Mease (1991) Nucl. Med. Bio. 18: 589-603. Alternatively, an anti-CD52 antibody may be labeled using “direct labeling” or a “direct labeling approach,” where a radionuclide is covalently attached directly to an antibody (typically via an amino acid residue). Preferred radionuclides are provided in Srivagtava and Mease (1991) supra. The indirect labeling approach is particularly preferred. See also, for example, labeled forms of anti-CD52 antibodies described in U.S. Pat. No. 6,015,542.

Anti-CD52 antibodies are typically provided by standard technique within a pharmaceutically acceptable buffer, for example, sterile saline, sterile buffered water, propylene glycol, combinations of the foregoing, etc. Methods for preparing parentally administrable agents are described in Remington's Pharmaceutical Sciences (18th ed.; Mack Pub. Co.: Eaton, Pa., 1990). See also, for example, International Publication No. WO 98/56418, which describes stabilized antibody pharmaceutical formulations suitable for use in the methods of the present invention.

A preferred anti-CD52 antibody is Alemtuzumab, also referred to herein as Campath®, and available from Berlex Laboratories, Richmond, Calif. 94804. Campath® is preferably administered intravenously or, as an alternative, subcutaneously (SC), as described by Lundin, et al. (2002) “Phase II trial of subcutaneous anti-CD52 monoconal antibody Alemtuzumab (Campath-1H) as first-line treatment for patients with B-cell chronic lymphocytic leukemia (B-cell),” Blood 100:768-773.

Definitions: IL-2. The term “an IL-2” as used herein refers to a lymphokine that is produced by normal peripheral blood lymphocytes and is present in the body at low concentrations. IL-2 was first described by Morgan et al. (1976) Science 193:1007-1008 and originally called T cell growth factor because of its ability to induce proliferation of stimulated T lymphocytes. It is a protein with a reported molecular weight in the range of 13,000 to 17,000 (Gillis and Watson (1980) J. Exp. Med. 159:1709) and has an isoelectric point in the range of 6-8.5. For purposes of the present invention, by the term “an IL-2” is intended IL-2 and variants thereof, whether native or obtained by recombinant techniques. The IL-2 may be the native polypeptide sequence, or can be a variant of a native IL-2 polypeptide as described herein below, so long as the variant IL-2 polypeptide retains IL-2 biological activity of interest as defined herein. Preferably an IL-2 polypeptide or variant thereof is derived from a human source, and includes human IL-2 that is recombinantly produced, such as recombinant human IL-2 polypeptides produced by microbial hosts, and variants thereof that retain the IL-2 biological activity of interest. Any pharmaceutical composition comprising an IL-2 as a therapeutically active component can be used to practice the present invention.

An IL-2 molecule useful in the methods of the invention may be a biologically active variant of a native IL-2. Such variant IL-2 polypeptides should retain the desired biological activity of the native polypeptide such that the pharmaceutical composition comprising the variant polypeptide has the same therapeutic effect as the pharmaceutical composition comprising the native polypeptide when administered to a subject. That is, the variant polypeptide will serve as a therapeutically active component in the pharmaceutical composition in a manner similar to that observed for the native polypeptide. Methods are available in the art for determining whether a variant polypeptide retains the desired biological activity, and hence serves as a therapeutically active component in the pharmaceutical composition. Biological activity can be measured using assays specifically designed for measuring activity of the native polypeptide or protein, including assays described in the present invention. Additionally, antibodies raised against a biologically active native polypeptide can be tested for their ability to bind to the variant polypeptide, where effective binding is indicative of a polypeptide having a conformation similar to that of the native polypeptide.

For purposes of the present invention, IL-2 biological activity of interest is the ability of an IL-2 to activate and/or expand normal T cells (CD and CD8) as well as natural killer (NK) cells. The NK cell numbers can be measured by standard flow cytometry analysis. NK-mediated antibody-dependent cellular cytotoxicity (ADCC) is also measured in a standard cytotoxicity assay, for example as described in Nagler, A., et al. (1989) J. Immunol. 143:3183-3191, and Thompson, J. (1987) et al., Cancer Research 47:4202-4207. The T-cell functional capability is tested by stimulation with recall antigens (PPD) and mitogens (PHA) and measured by ³H-thymidine incorporation and at the single cell level by ELISPOT assay. Thus, an IL-2 (for example, a mutein of human IL-2) for use in the methods of the present invention will activate and/or expand normal T cells, and also natural killer (NK) cells to mediate antibody dependent cellular cytotoxicity (ADCC). NK cells mediate spontaneous or natural cytotoxicity against certain cell targets referred to as “NK-cell sensitive” targets, such as the human erythroleukemia K562 cell line. Following activation by an IL-2, NK cells acquire LAK activity. Such LAK activity can be assayed by the ability of IL-2-activated NK cells to kill a broad variety of tumor cells and other “NK-insensitive” targets, such as the Daudi B-cell lymphoma line, that are normally resistant to lysis by resting (nonactivated) NK cells. Similarly, ADCC activity can be assayed by the ability of IL-2 activated NK cells to lyse “NK-insensitive” target cells, such as Daudi B-cell lymphoma line, or other target cells not readily lysed by resting NK cells in the presence of optimal concentrations of relevant tumor cell specific antibodies. Methods for generating and measuring cytotoxic activity of NK/LAK cells and ADCC are known in the art. See, for example, Current Protocols in Immunology: Immunologic Studies in Humans, Supplement 17, Unit 7.7, 7.18, and 7.27 (John Wiley & Sons, Inc., 1996).

For purposes of the present invention, NK cells activated by an IL-2 for use in the methods of the present invention demonstrate a specific lysing activity of NK-insensitive cells in the presence (ADCC activity) or absence (LAK activity) of antibody, more particularly NK-insensitive Daudi cells in the presence of B-cell specific antibodies including rituximab, that is at least about 20% greater, or at least about 25%, or 30%, or 35%, or 40% greater than baseline lysing activity of resting NK cells (i.e., nonactivated) as measured using effector to target ratios between 12.5 to 50:1 in a standard 4-hr ⁵¹Cr-release cytotoxicity assay (see Current Protocols in Immunology: Immunologic Studies in Humans, Unit 7.7, Supplement 17, Section 17.18.1 (John Wiley & Sons, Inc., 1996)). In some embodiments, the specific lysing activity of NK cells activated by an IL-2 is at least about 45% greater, at least about 50% greater, at least about 55% greater, or at least about 60% greater than baseline lysing activity of resting NK cells when measured as noted above.

Suitable biologically active variants of native or naturally occurring IL-2 can be fragments, analogues, and derivatives of that polypeptide. By “fragment” is intended a polypeptide consisting of only a part of the intact polypeptide sequence and structure, and can be a C-terminal deletion or N-terminal deletion of the native polypeptide. By “analogue” is intended an analogue of either the native polypeptide or of a fragment of the native polypeptide, where the analogue comprises a native polypeptide sequence and structure having one or more amino acid substitutions, insertions, or deletions. “Muteins,” such as those described herein, and peptides having one or more peptoids (peptide mimics) are also encompassed by the term analogue (see International Publication No. WO 91/04282). By “derivative” is intended any suitable modification of the native polypeptide of interest, of a fragment of the native polypeptide, or of their respective analogues, such as glycosylation, phosphorylation, polymer conjugation (such as with polyethylene glycol), or other addition of foreign moieties, so long as the desired biological activity of the native polypeptide is retained. Methods for making polypeptide fragments, analogues, and derivatives are generally available in the art.

For example, amino acid sequence variants of the polypeptide can be prepared by mutations in the cloned DNA sequence encoding the native polypeptide of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (Cold Spring Harbor, N.Y.); U.S. Pat. No. 4,873,192; and the references cited therein. Guidance as to appropriate amino acid substitutions that do not affect biological activity of the polypeptide of interest may be found in the model of Dayhoff et al. (1978) in Atlas of Protein Sequence and Structure (Natl. Biomed. Res. Found., Washington, D.C.).

Conservative substitutions, such as exchanging one amino acid with another having similar properties, may be preferred. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr.

In constructing variants of an IL-2 polypeptide of interest, modifications are made such that variants continue to possess the desired activity. Obviously, any mutations made in the DNA encoding the variant polypeptide must not place the sequence out of reading frame and preferably will not create complementary regions that could produce secondary mRNA structure. See EP Patent Application Publication No. 75,444.

Preferably, naturally or non-naturally occurring variants of a reference IL-2 molecule have amino acid sequences that have at least 70% or 75% sequence identity, preferably at least 80% or 85% sequence identity, more preferably at least 90%, 91%, 92%, 93%, 94% or 95% sequence identity to the amino acid sequence for the reference IL-2 molecule, for example, native human IL-2, or to a shorter portion of the reference IL-2 molecule. More preferably, the molecules share at least 96%, 97%, 98% or 99% sequence identity. For purposes of the present invention, percent sequence identity is determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman (1981) Adv. Appl. Math. 2:482-489. A variant may, for example, differ from the reference IL-2 molecule by as few as 1 to 15 amino acid residues, as few as 1 to 10 amino acid residues, such as 6-10, as few as 5, as few as 4, 3, 2, or even 1 amino acid residue.

With respect to optimal alignment of two amino acid sequences, the contiguous segment of the variant amino acid sequence may have additional amino acid residues or deleted amino acid residues with respect to the reference amino acid sequence. The contiguous segment used for comparison to the reference amino acid sequence will include at least 20 contiguous amino acid residues, and may be 30, 40, 50, or more amino acid residues. Corrections for sequence identity associated with conservative residue substitutions or gaps can be made (see Smith-Waterman homology search algorithm).

The precise chemical structure of a polypeptide having an IL-2 activity depends on a number of factors. As ionizable amino and carboxyl groups are present in the molecule, a particular polypeptide may be obtained as an acidic or basic salt, or in neutral form. All such preparations that retain their biological activity when placed in suitable environmental conditions are included in the definition of polypeptides having an IL-2 activity as used herein. Further, the primary amino acid sequence of the polypeptide may be augmented by derivatization using sugar moieties (glycosylation) or by other supplementary molecules such as lipids, phosphate, acetyl groups and the like. It may also be augmented by conjugation with saccharides. Certain aspects of such augmentation are accomplished through post-translational processing systems of the producing host; other such modifications may be introduced in vitro. In any event, such modifications are included in the definition of an IL-2 polypeptide used herein so long as IL-2 activity of the polypeptide is not destroyed. It is expected that such modifications may quantitatively or qualitatively affect the activity, either by enhancing or diminishing the activity of the polypeptide, in the various assays. Further, individual amino acid residues in the chain may be modified by oxidation, reduction, or other derivatization, and the polypeptide may be cleaved to obtain fragments that retain activity. Such alterations that do not destroy activity do not remove the polypeptide sequence from the definition of an IL-2 as used herein.

The art provides substantial guidance regarding the preparation and use of polypeptide variants. In preparing an IL-2, one of skill in the art can readily determine which modifications to the native protein nucleotide or amino acid sequence will result in a variant that is suitable for use as a therapeutically active component of a pharmaceutical composition used in the methods of the present invention.

An IL-2 for use in the methods of the present invention may be from any source, but preferably is a recombinant IL-2. By “recombinant IL-2” is intended an interleukin-2 that has comparable biological activity to native-sequence IL-2 and that has been prepared by recombinant DNA techniques as described, for example, by Taniguchi et al. (1983) Nature 302:305-310 and Devos (1983) Nucleic Acids Research 11:4307-4323 or mutationally altered IL-2 as described by Wang et al. (1984) Science 224:1431-1433. In general, the gene coding for an IL-2 is cloned and then expressed in transformed organisms, preferably a microorganism, for example E. coli, as described herein. The host organism expresses the foreign gene to produce an IL-2 under expression conditions. Synthetic recombinant IL-2 can also be made in eukaryotes, such as yeast or human cells. Processes for growing, harvesting, disrupting, or extracting an IL-2 from cells are known in the art as evidenced by, for example, U.S. Pat. Nos. 4,604,377; 4,738,927; 4,656,132; 4,569,790; 4,748,234; 4,530,787; 4,572,798; 4,748,234; and 4,931,543.

For examples of IL-2 proteins, see European Patent (EP) Publication No. EP 136,489 (which discloses one or more of the following alterations in the amino acid sequence of naturally occurring IL-2: Asn26 to Gln26; TrpI21 to Phe121; Cys58 to Ser58 or Ala58, CysI05 to Ser105 or Ala105; Cys125 to Ser125 or Ala125; deletion of all residues following Arg 120; and the Met-1 forms thereof); and the recombinant IL-2 muteins described in European Patent Application No. 83306221.9, filed Oct. 13, 1983 (published May 30, 1984 under Publication No. EP 109,748), which is the equivalent to Belgian Patent No. 893,016, and commonly owned U.S. Pat. No. 4,518,584 (which disclose recombinant human IL-2 mutein wherein the cysteine at position 125, numbered in accordance with native human IL-2, is deleted or replaced by a neutral amino acid; alanyl-ser125-IL-2; and des-alanayl-ser125-IL2). See also U.S. Pat. No. 4,752,585 (which discloses the following variant IL-2 proteins: ala104 ser125 IL-2, ala104 IL-2, ala104 ala125 IL-2, val 104 ser125 IL-2, val104 IL-2, val104 ala125 IL-2, des-ala1 ala104 ser125 IL-2, des-ala1 ala104 IL-2, des-ala1 ala104 ala125 IL-2, des-ala1 val104 ser125 IL-2, des-ala1 val104 IL-2, desala1 val104 ala125 IL-2, des-ala1 des-pro2 ala104 ser125 IL-2, des-ala1 des-pro2 ala104 IL-2, des-ala1 des-pro2 ala104 ala125 IL-2, des-ala1 des-pro2 val104 ser125 IL-2, des-ala1 des-pro2 val104 IL-2, des-ala1 des-pro2 val104 ala125 IL-2, des-ala1 des-pro2 des-thr3 ala104 ser125 IL-2, des-ala1 des-pro2 des-thr3 ala104 IL-2, desala1 des-pro2 des-thr3 ala104 ala125 IL-2, des-ala1 des-pro2 des-thr3 val104 ser125 IL-2, des-ala1 des-pro2 des-thr3 val104 IL-2, des-ala1 des-pro2 des-thr3 val104 ala125 mL-2, des-ala1 des-pro2 des-thr3 des-ser4 ala104 ser125 IL-2, des-ala1 des pro2 des-thr3 des-ser4 ala104 mL-2, des-ala1 des-pro2 des-thr3 des-ser4 ala104 ala125 mL-2, des-ala1 des-pro2 des-thr3 des-ser4 val104 ser125 mL-2, des-ala1 des-pro2 desthr3 des-ser4 val104 IL-2, des-ala1 des-pro2 des-thr3 des-ser4 val104 ala125 IL-2, des-ala1 des-pro2 des-thr3 des-ser4 des-ser5 ala104 ser125 IL-2, des-ala1 des-pro2 des-thr3 des-ser4 des-ser5 ala104 IL-2, des-ala1 des-pro2 des-thr3 des-ser4 des-ser5 ala104 ala125 mL-2, des-ala1 des-pro2 des-thr3 des-ser4 des-ser5 val104 ser125 mL-2, des-ala1 des-pro2 des-thr3 des-ser4 des-ser5 val 104 IL-2, des-ala1 des-pro2 des-thr3 des-ser4 des-ser5 val104 ala125 IL-2, des-ala1 des-pro2 des-thr3 des-ser4 des-ser5 des-ser6 ala104 ala125 IL-2, des-ala1 des-pro2 des-thr3 des-ser4 des-ser5 des-ser6 ala104 IL-2, des-ala1 des-pro2 des-thr3 des-ser4 des-ser5 des-ser6 ala104 ser125 IL2, des-ala1 des-pro2 des-thr3 des-ser4 des-ser5 des-ser6 val104 ser125 IL-2, des-ala1 des-pro2 des-thr3 des-ser4 des-ser5 des-ser6 val104 IL-2, and des-ala 1 des-pro2 desthr3 des-ser4 des-ser5 des-ser6 val104 ala125 IL-2) and U.S. Pat. No. 4,931,543 (which discloses the IL-2 mutein des-alanyl-I, serine-125 human IL-2 used in the examples herein, as well as the other IL-2 muteins).

Also see European Patent Publication No. EP 200,280 (published Dec. 10, 1986), which discloses recombinant IL-2 muteins wherein the methionine at position 104 has been replaced by a conservative amino acid. Examples include the following muteins: ser4 des-ser5 ala104 IL-2; des-ala1 des-pro2 des-thr3 des-ser4 des ser5 ala104 ala125 IL-2; des-ala1 des-pro2 des-thr3 des-ser4 des-ser5 glu104 ser125 IL-2; des-ala1 des-pro2 des-thr3 des-ser4 des-ser5 glu104 IL-2; des-ala1 des-pro2 desthr3 des-ser4 des-ser5 glu104 ala125 IL-2; des-ala1 des-pro2 des-thr3 des-ser4 des-ser5 des-ser6 ala104 ala125 IL-2; des-ala1 des-pro2 des-thr3 des-ser4 des-ser5 des-ser6 ala104 IL-2; des-ala1 des-pro2 des-thr3 des-ser4 des-ser5 des-ser6 ala104 ser125 IL-2; des-ala1 des-pro2 des-thr3 des-ser4 des-ser5 des-ser6 glu104 ser125 IL-2; desala1 des-pro2 des-thr3 des-ser4 des-ser5 des-ser6 glu104 IL-2; and des-ala1 des-pro2 des-thr3 des-ser4 des-ser5 des-ser6 glu104 ala125 IL-2. See also European Patent Publication No. EP 118,617 and U.S. Pat. No. 5,700,913, which disclose unglycosylated human IL-2 variants bearing alanine instead of native IL-2's methionine as the N-terminal amino acid; an unglycosylated human IL-2 with the initial methionine deleted such that proline is the N-terminal amino acid; and an unglycosylated human IL-2 with an alanine inserted between the N-terminal methionine and proline amino acids.

Other IL-2 muteins include the those disclosed in WO 99/60128 (substitutions of the aspartate at position 20 with histidine or isoleucine, the asparagine at position 88 with arginine, glycine, or isoleucine, or the glutamine at position 126 with leucine or gulatamic acid), which reportedly have selective activity for high affinity IL-2 receptors expressed by cells expressing T cell receptors in preference to NK cells and reduced IL-2 toxicity; the muteins disclosed in U.S. Pat. No. 5,229,109 (substitutions of arginine at position 38 with alanine, or substitutions of phenylalanine at position 42 with lysine), which exhibit reduced binding to the high affinity IL-2 receptor when compared to native IL-2 while maintaining the ability to stimulate LAK cells; the muteins disclosed in International Publication No. WO 00/58456 (altering or deleting a naturally occurring (x)D(y) sequence in native IL-2 where D is aspartic acid, (x) is leucine, isoleucine, glycine, or valine, and (y) is valine, leucine or serine), which are claimed to reduce vascular leak syndrome; the IL-2 pl-30 peptide disclosed in International Publication No. WO 00/04048 (corresponding to the first 30 amino acids of IL-2, which contains the entire a-helix A of IL-2 and interacts with the b chain of the IL-2 receptor), which reportedly stimulates NK cells and induction of LAK cells; and a mutant form of the IL-2 pl-30 peptide also disclosed in WO 00/04048 (substitution of aspartic acid at position 20 with lysine), which reportedly is unable to induce vascular bleeds but remains capable of generating LAK cells. Additionally, an IL-2 can be modified with polyethylene glycol to provide enhanced solubility and an altered pharmokinetic profile (see U.S. Pat. No. 4,766,106).

The term “an IL-2” as used herein is also intended to include IL-2 fusions or conjugates comprising an IL-2 fused to a second protein or covalently conjugated to polyproline or a water-soluble polymer to reduce dosing frequencies or to improve IL-2 tolerability. For example, an IL-2 can be fused to human albumin or an albumin fragment using methods known in the art (see WO 01/79258). Alternatively, an IL-2 can be covalently conjugated to polyproline or polyethylene glycol homopolymers and polyoxyethylated polyols, wherein the homopolymer is unsubstituted or substituted at one end with an alkyl group and the polyol is unsubstituted, using methods known in the art (see, for example, U.S. Pat. Nos. 4,766,106, 5,206,344, and 4,894,226).

Overview: IL-2 and anti-CD52 therapy. The present invention provides concurrent therapy with an IL-2 and an anti-CD52 antibody for the treatment of any type of cancer whose unabated proliferating cells express the CD52 surface antigen. Thus, for example, where a cancer is associated with aberrant T-cell proliferation, and the aberrant T-cell population expresses the CD52 surface antigen, concurrent therapy in accordance with the methods of the invention would provide a positive therapeutic response with respect to treatment of that cancer. In such a situation, however, the possibility of stimulating not only normal but also malignant T-cells by an IL-2 must be considered.

In accordance with the methods of the present invention, an IL-2 and an anti-CD52 antibody are used in combination to promote a stimulatory effect on normal T-cells which would reduce the risk of severe infections. A positive therapeutic response with respect to chronic lymphocytic leukemia may also achieved. By “positive therapeutic response” is intended an improvement in the disease in association with the combined therapeutic activity of these agents, and/or an improvement in the symptoms associated with the disease. Thus, for example, an improvement in the disease may be characterized as a complete response. By “complete response” is intended an absence of clinically detectable disease with normalization of any previously abnormal blood cell counts, radiographic studies (such as CT scan), bone marrow, and cerebrospinal fluid (CSF). Such a response must persist for at least one month following treatment according to the methods of the invention. A complete response can be unconfirmed if no repeat evaluation of tumor status is done at least one month after the initial response is evaluated. Alternatively, an improvement in the disease may be categorized as being a partial response. By “partial response” is intended at least a substantial decrease in all measurable tumor burden (i.e., the number of tumor cells present in the subject) in the absence of new lesions and persisting for at least one month. Such a response is applicable to measurable tumors only. In addition to these positive therapeutic responses, the subject undergoing concurrent therapy with these two therapeutic agents may experience the beneficial effect of an improvement in the symptoms associated with the disease. Thus the subject may experience a decrease in the so-called B symptoms, i.e., night sweats, fever, weight loss, and/or urticaria.

Promotion of a positive therapeutic response with respect to a CLL in a human subject is achieved via concurrent therapy with both an anti-CD52 antibody and an IL-2. By “concurrent therapy” is intended presentation of an IL-2 and an anti-CD52 antibody to a human subject such that the therapeutic effect of the combination of both substances is caused in the subject undergoing therapy. Concurrent therapy may be achieved by administering at least one therapeutically effective dose of a pharmaceutical composition comprising an IL-2 and at least one therapeutically effective dose of a pharmaceutical composition comprising an anti-CD52 antibody according to a particular dosing regimen. For example, in accordance with the methods of the present invention, concurrent therapy is achieved by administering the recommended total weekly doses of a pharmaceutical composition comprising an IL-2 in combination with the recommended therapeutically effective doses of a pharmaceutical composition comprising an anti-CD52 antibody, each being administered according to a particular dosing regimen. By “therapeutically effective dose or amount” is intended an amount of the therapeutic agent that, when administered with a therapeutically effective dose or amount of the other therapeutic agent, brings about a positive therapeutic response with respect to treatment of a leukemia such as CLL. Administration of the separate pharmaceutical compositions can be at the same time (i.e., simultaneously) or at different times (i.e., sequentially, in either order, on the same day, or on different days), so long as the therapeutic effect of the combination of both substances is caused in the subject undergoing therapy. According to the present invention the combination allows the partial or total eradication of leukemia cells by the antibody treatment, with an IL-2 providing for preservation of T-cell counts and functions.

Routes of administration. The separate pharmaceutical compositions comprising these therapeutic agents as therapeutically active components may be administered using any acceptable method known in the art. Thus, for example, the pharmaceutical composition comprising an IL-2 can be administered by any form of injection, including intravenous (IV), intramuscular (IM), or subcutaneous (SC) injection. In the protocols described in the Examples herein, the pharmaceutical composition comprising an IL-2 is administered by SC injection. In other embodiments of the invention, the pharmaceutical composition comprising an IL-2 is a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release IL-2 pharmaceutical composition. The pharmaceutical composition comprising an anti-CD52 antibody is preferably administered, for example, subcutaneously as described further herein. In the event that an anti-CD52 antibody is administered intravenously, the pharmaceutical composition comprising an anti-CD52 antibody can be administered by infusion over a period of about 30 minutes or 1 hour, to about 10 hours. In some embodiments, infusion of the antibody occurs over a period of about 2 to about 8 hours, over a period of about 3 to about 7 hours, over a period of about 4 to about 6 hours, or over a period of about 6 hours, depending upon the anti-CD52 antibody being administered.

Concurrent therapy. Concurrent therapy with both of these therapeutic agents in the manner set forth herein provides for an improved safety and possibly also a greater therapeutic effectiveness than can be achieved using either of these agents alone, resulting in a positive therapeutic response that is improved with respect to that observed with either agent alone. In particular, treatment with an IL-2 allows administration of an anti-CD52 antibody with less serious, even life-threatening, complications and also for a longer duration as fewer subjects are expected to be subject to premature withdrawal from anti-CD52 antibody therapy due to infections than without an IL-2.

The amount of an anti-CD52 antibody to be administered in combination with an amount of an IL-2, and the amount of either of these therapeutic agents needed to potentiate the effectiveness of the other therapeutic agent, are readily determined by one of ordinary skill in the art without undue experimentation given the disclosure set forth herein. Factors influencing the respective amount of an IL-2 to be administered in combination with a given amount of an anti-CD52 antibody in accordance with the dosing regimens disclosed herein include, but are not limited to, the mode of administration, the particular leukemia undergoing therapy, the severity of the disease, the history of the disease, and the age, height, weight, health, and physical condition of the individual undergoing therapy. Similarly, these factors will influence the necessity for repeated exposure to combination IL-2/anti-CD52 antibody therapy in the manner set forth herein. Generally, a higher dosage of the antibody agent is preferred with increasing weight of the subject undergoing therapy.

In accordance with the methods of the present invention, the human subject undergoing treatment with therapeutically effective doses of an anti-CD52 antibody according to a weekly, twice-weekly, or thrice-weekly dosing regimen described herein below is also administered an IL-2 according to a constant IL-2 dosing regimen or according to a two-level IL-2 dosing regimen. An important aspect of the invention is an overlapping period of time during which both therapeutic agents are being administered to the subject, each according to the particular dosing regimen disclosed herein. On those days where both an anti-CD52 antibody and an IL-2 are scheduled to be administered to the subject, these therapeutic agents can be administered either at the same time (i.e., simultaneous administration) or at different times (i.e., sequential administration, in either order). In one such embodiment, the dose of an anti-CD52 antibody is administered first, followed by administration of the dose of an IL-2 within about 10 minutes to about 4 hours of the completion of administering the dose of an anti-CD52 antibody, such as within about 10, 15, 20, 25, 30, 45, 60, 90, 120, 150, 180, 210, or 240 minutes.

Concurrent therapy: initiation of treatment. The constant or two-level IL-2 dosing regimen and an anti-CD52 antibody administration begin concurrently on the same day, either at the same time (i.e., simultaneous administration) or at different times (i.e., sequential administration, in either order). Thus, for example, in one embodiment where concurrent therapy with these two therapeutic agents begins on day 1 of a treatment period, a first therapeutically effective dose of an anti-CD52 antibody and a first dose of an IL-2 would both be administered on day 1 of this treatment period. In one such embodiment, the dose of an anti-CD52 antibody is administered first, followed by administration of the dose of an IL-2 within about 10 minutes to about 4 hours of the completion of administering the dose of an anti-CD52 antibody, such as within about 10, 15, 20, 25, 30, 45, 60, 90, 120, 150, 180, 210, or 240 minutes.

In alternative embodiments, a first therapeutically effective dose of an anti-CD52 antibody is administered to the subject, for example, on day 1 of a treatment period, and the constant or two-level IL-2 dosing regimen is initiated by administering a first dose of an IL-2 within 10 days of administering the first therapeutically effective dose of an anti-CD52 antibody. In such embodiments, preferably the constant or two-level IL-2 dosing regimen is initiated by administering a first dose of an IL-2 within 7 days of administering the first therapeutically effective dose of an anti-CD52 antibody, such as within 1, 2, 3, 4, 5, 6, or 7 days.

Concurrent therapy: dosing frequency. Depending upon the severity of the disease, the patient's health, and prior history of the patient's disease, one or more cycles of a constant IL-2 dosing regimen or a two-level IL-2 dosing regimen can be administered concurrently with anti-CD52 antibody therapy, where therapeutically effective doses of the antibody are administered weekly, twice-weekly, or thrice-weekly.

In accordance with the methods of the present invention, a therapeutically effective dose of an anti-CD52 antibody is preferably administered thrice weekly, or is administered once or twice weekly, in combination with one or more cycles of a constant IL-2 dosing regimen or in combination with one or more cycles of a two-level IL-2 dosing regimen. The duration of anti-CD52 antibody administration and the duration of any given cycle of either of the IL-2 dosing regimens will depend upon the subject's overall health, history of disease progression, and tolerance of the particular anti-CD52 antibody/IL-2 administration protocol. Generally, therapeutically effective doses of anti-CD52 antibody are administered weekly (i.e., once a week), twice-weekly, or thrice-weekly for 8-16 weeks, for example, 8, 9, 10, 11, 12, 13, 14, 15, or 16 weeks.

In the context of anti-CD52 antibody administration, a “twice weekly” or “two times per week” dosing schedule is intended to mean two therapeutically effective doses of an anti-CD52 antibody are administered within a 7-day period, beginning on day 1 of the first week of anti-CD52 antibody administration, allowing for a minimum of 72 hours between doses and a maximum of 96 hours between doses. Thus, for example, if twice-weekly anti-CD52 antibody administration begins on day 1 of any given week of antibody administration, the second dose for that week can be administered on day 3 or day 4 of that week of the treatment period, with the next therapeutically effective dose of an IL-2 being administered on day 1 of the next week of antibody administration.

Similarly, in the context of anti-CD52 antibody administration, a “three-times-a-week,” “thrice weekly,” or “three times per week” dosing schedule is intended three therapeutically effective doses are administered to the subject within a 7-day period, beginning on day 1 of the first week of anti-CD52 antibody administration, allowing for at least 24 hours between doses and a maximum of 72 hours between doses. Thus, for example, where the first therapeutically effective dose of an anti-CD52 antibody is administered on day 1 of a given week of a treatment period, the second dose can be administered on day 2, 3, 4, 5, or 6 of that week, and the third dose can be administered on day 3, 4, 5, 6, or 7 of that week, so long as about 24 hours occur between administration of the second and third doses. In one embodiment, the three therapeutically effective doses of an anti-CD52 antibody are administered to the subject within a 7-day period, allowing for at least 25 hours between doses and a maximum of 72 hours between doses, for example on day 1, day 3, and day 5 of each week of antibody administration.

Duration of IL-2 treatment. The duration of IL-2 administration is a function of the IL-2 dosing regimen used. Generally, an IL-2 is administered according to the disclosed protocols, and a subject can repeat one or more cycles of a constant or two-level IL-2 dosing regimen as needed, unless IL-2 toxicity symptoms develop. Should such toxicity symptoms develop, the subject can be taken off of IL-2 dosing until complete resolution of any observed toxicity symptoms. Such IL-2 toxicity responses include but are not limited to, chronic fatigue, nausea, hypotension, fever, chills, weight gain, pruritis or rash, dysprea, azotemia, confusion, thrombocytopenia, myocardial infarction, gastrointestinal toxicity, and vascular leak syndrome (see, for example, Allison et al. (1989) J. Clin. Oncol. 7(1):75-80).

Constant IL-2 dosing regimen. In some embodiments of the invention, the subject undergoing concurrent therapy with these two therapeutic agents is administered a constant IL-2 dosing regimen. By “constant L-2 dosing regimen” is intended the subject undergoing concurrent therapy with an IL-2 and an anti-CD52 antibody is administered a constant total weekly dose of an IL-2 over the course of any given cycle of IL-2 administration. One complete cycle of a constant IL-2 dosing regimen comprises administering a constant total weekly dose of an IL-2 for a period of about 10 weeks to about 20 weeks, such as about 10, 11, 12, 13, 14, 15, 16 17, 18, 19, or 20 weeks, at which time IL-2 dosing is discontinued. The duration of the constant IL-2 dosing regimen preferably lasts for 2-6 weeks after cessation of anti-CD52 antibody administration, for example, 2, 3, 4, 5, or 6 weeks following cessation of anti-CD52 antibody administration.

At the discretion of the managing physician, the subject undergoing concurrent therapy with an anti-CD52 antibody and a constant IL-2 dosing regimen can continue receiving the constant total weekly dose of an IL-2 for an extended period of time beyond 20 weeks, for example, for an additional 1-8 weeks, providing the constant IL-2 dosing protocol is well tolerated and the subject is exhibiting minimal signs of IL-2 toxicity symptoms.

In some embodiments, the first therapeutically effective dose of an anti-CD52 antibody is administered beginning on day 1 of a treatment period, and the constant IL-2 dosing regimen is initiated by administering a first dose of an IL-2 beginning on day 8 of that treatment period. In one such embodiment, the duration of anti-CD52 antibody administration is 8-12 weeks, including 8, 9, 10, 11, or 12 weeks, beginning on day 1 of a treatment period, and a first cycle of a constant IL-2 dosing regimen is initiated on day 8 of that treatment period and has a duration that lasts 2-4 weeks longer than the duration of anti-CD52 antibody administration.

In preferred embodiments, a first dose of an anti-CD52 antibody is administered beginning on day 1 of a treatment period, with subsequent antibody doses being administered according to a weekly, twice-weekly, or thrice-weekly dosing regimen for a total of 12 weeks, and the constant IL-2 dosing regimen is initiated on day 8 of that treatment period and has a duration that lasts 4 weeks longer than anti-CD52 antibody administration (i.e., through week 16 of the treatment period). In one such embodiment, an anti-CD52 antibody is administered according to a thrice-weekly dosing schedule.

Two-level IL-2 dosing regimen. In other embodiments of the invention, concurrent therapy with these two therapeutic agents comprises a “two-level IL-2 dosing regimen.” By “two-level IL-2 dosing regimen” is intended the subject undergoing concurrent therapy with an IL-2 and an anti-CD52 antibody is administered an IL-2 during two time periods of IL-2 dosing, which have a combined duration of about 10 weeks to about 20 weeks, including 10, 11, 12, 13, 14, 15, 16, 17, 18, 19, or 20 weeks. In one embodiment, the two-level IL-2 dosing regimen has a combined duration of about 10 weeks to about 16 weeks; in other embodiments, the two-level IL-2 dosing regimen has a combined duration of about 12 weeks to about 18 weeks; in still other embodiments, the two-level IL-2 dosing regimen has a combined duration of about 14 weeks to about 20 weeks. Preferably the combined duration of the two-level IL-2 dosing regimen is such that IL-2 administration continues for 2-6 weeks after cessation of anti-CD52 antibody administration, for example, 2, 3, 4, 5, or 6 weeks following cessation of anti-CD52 antibody administration.

The total weekly dose of an IL-2 that is to be administered during the first and second time periods of the two-level IL-2 dosing regimen is chosen such that a higher total weekly dose of an IL-2 is given during the first time period and a lower total weekly dose of an IL-2 is given during the second time period. The duration of the individual first and second time periods of the two-level IL-2 dosing regimen can vary, depending upon the health of the individual and history of disease progression. Generally, the subject is administered higher total weekly doses of an IL-2 for at least 1 week out of the 10-week to 20-week two-level IL-2 dosing regimen. In one embodiment, higher total weekly doses of an IL-2 are administered during the first half of the two-level IL-2 dosing regimen, with lower total weekly doses being administered during the second half of the two-level IL-2 dosing regimen. Thus, for example, where the two-level IL-2 dosing regimen has a combined duration of 12 weeks, the higher total weekly doses of an IL-2 would be administered for the first 6 weeks of IL-2 dosing, and the lower total weekly doses of an IL-2 would be administered for the second 6 weeks of IL-2 dosing.

Exemplary dosing regimens. Though specific dosing regimens are disclosed herein below, it is recognized that the invention encompasses any administration protocol that provides for concurrent therapy with an anti-CD52 antibody and a two-level IL-2 dosing regimen that provides for initial exposure to higher total weekly doses of an IL-2, and subsequent exposure to lower total weekly doses of an IL-2, although in some embodiments the initial exposure may utilize a lower dose and subsequent exposure may utilize a higher dose. While not being bound by theory, it is believed that administering a higher dose of an IL-2 during the initial stages of IL-2 dosing provides for an initial preservation of T-cell counts that can be maintained by a lower dose during the subsequent weeks of IL-2 dosing. As IL-2 side effects are dose-related, the lowered dose will increase tolerability during the extended treatment period.

Thus, the methods of the invention contemplate treatment regimens where a therapeutically effective dose of an anti-CD52 antibody is administered according to a weekly, twice-weekly, or thrice-weekly dosing schedule for about 8 weeks to about 14 weeks, in combination with a two-level IL-2 dosing regimen having a combined duration that is about 10 weeks to about 20 weeks, such that IL-2 dosing continues for about 2 weeks to about 6 weeks longer than the duration of anti-CD52 antibody administration.

For example, in one embodiment, a therapeutically effective dose of an anti-CD52 antibody is administered first, for example, on day 1 of a treatment period, followed by initiation of the two-level IL-2 dosing regimen within 10 days, preferably within 7 days of the first administration of the anti-CD52 antibody, for example, within 1, 2, 3, 4, 5, 6, or 7 days. During the two-level IL-2 dosing regimen, a higher total weekly dose of an IL-2 is administered in the first time period of the two-level IL-2 dosing regimen, for example, over the first 1-5 weeks, or over the first 1-10 weeks, of IL-2 administration, and lower total weekly doses of an IL-2 are administered during the second time period of the two-level IL-2 dosing regimen (i.e., over the remaining course of the two-level IL-2 dosing regimen).

In some embodiments, the methods of the invention provide for thrice-weekly administration of a therapeutically effective dose of a pharmaceutical composition comprising an anti-CD52 antibody beginning on day 1 of a treatment period and continuing over a period of 12 weeks in combination with a two-level IL-2 dosing regimen having a combined duration of 16 weeks. In these embodiments, the two-level IL-2 dosing regimen is initiated beginning on day 3, 4, 5, 6, 7, 8, 9, or 10 of the same treatment period. In one such embodiment, the two-level IL-2 dosing regimen is initiated on day 8 of the treatment period, and the first time period of the two-level IL-2 dosing regimen has a duration of about 1 week to about 8 weeks, such as 2, 3, 4, 5, 6, 7, or 8 weeks out of the 16-week period of IL-2 administration, with the lower IL-2 dose being administered during the remainder of the 16-week period of IL-2 administration.

The exemplary dosing regimens discussed above relate to the concurrent therapy with an anti-CD52 antibody and an IL-2, wherein the IL-2 is administered to enhance the effect of anti-CD52 treatment. Following the concurrent therapy, a regimen of IL-2 treatment alone may be provided to restore the T-cell count. This regimen consists of administering an IL-2 twice a day for 5 consecutive days, followed by several weeks off of IL-2, for example, seven weeks off. (No IL-2 treatment is given during these weeks.) Each dose of IL-2 is 2-10 MIU, more usually 2-7.5 MIU, and an exemplary dose is 4.5 MIU, administered subcutaneously. After the seven weeks (eight weeks total cycle time) another cycle of IL-2 treatment alone can be given, to restore T-cell counts. By “IL-2 treatment alone” is meant the anti-CD52 antibody is not administered during the five consecutive days of IL-2 treatment.

Interruption of IL-2 dosing. The methods of the present invention also contemplate embodiments where a subject undergoing anti-CD52 antibody administration according to the dosing schedule recommended herein in combination with administration of one or more cycles of a two-level IL-2 dosing regimen is given a “drug holiday” or a time period off from IL-2 dosing, or from the IL-2 dosing and the anti-CD52 antibody dosing, between the conclusion of the first time period of any given cycle of the two-level IL-2 dosing regimen and the initiation of the second time period of that particular cycle of the two-level IL-2 dosing regimen. In these embodiments, the two-level IL-2 dosing regimen is interrupted such that IL-2 dosing is withheld for a period of about 1 week to about 4 weeks following conclusion of the first time period of a given cycle of the two-level IL-2 dosing regimen during which the higher total weekly dose has been administered. During this time period off of IL-2 dosing, the subject may continue to receive a therapeutically effective dose of an anti-CD52 antibody according to the dosing schedule recommended herein (i.e., once per week, or twice- or thrice-weekly), or alternatively, anti-CD52 antibody administration can also be stopped. The length of this interruption in IL-2 dosing will depend upon the health of the subject, history of disease progression, and responsiveness of the subject to the initial IL-2/antibody therapy received during the first time period of any given cycle of the two-level IL-2 dosing regimen. Generally, IL-2 dosing is interrupted for a period of about 1 week to about 4 weeks, at which time the subject is administered the second time period of the two-level IL-2 dosing regimen. In order to complete any given cycle of a two-level IL-2 dosing regimen, a subject must be administered both the first period of higher total weekly dosing and the second period of lower total weekly dosing.

During this drug holiday (i.e., time period off of IL-2 administration, or time period off of IL-2 and anti-CD52 antibody administration), lymphocyte subsets are monitored, for example, by FACS analysis, to determine when the two-level IL-2 dosing regimen, or the two-level IL-2 dosing regimen and anti-CD52 antibody administration, are to be resumed. Of particular interest are NK-cell counts and T-cell counts. In general, NK (CD3-56+), NK-T (CD3+56+), and T cell (CD3+4+, CD3+8+) cell numbers should be determined by flow cytometry analysis. In this manner, NK, NK-T and T-cell counts are measured bi-weekly or monthly during the two-dose IL-2 dosing regimen, and at the conclusion of the first time period of the two-level IL-2 dosing regimen before the drug holiday is initiated. Following discontinuance of the 12 dosing, which may or may not include discontinuance of anti-CD52 antibody administration, NK-cell counts and NK-T cell counts and T-cell counts are then measured at regular intervals, once per week or twice per week thereafter, preferably once per week. Two consecutive measurements showing a decrease in T-cell count to less than about 80% of the maximum count achieved during the initial period of IL-2 dosing is indicative of the need to reinstate IL-2 administration, or IL-2 and anti-CD52 antibody administration. A further indicator is a level of CD4+ cell counts below about <100/μl. At this time, the subject is administered the second time period of the two-level IL-2 dosing regimen, where lower total weekly doses of an IL-2 are administered in combination with the weekly administration of therapeutically effective doses of an anti-CD52 antibody.

Subsequent courses of IL-2/anti-CD52 antibody therapy. Where a subject undergoing therapy in accordance with the previously mentioned dosing regimens exhibits a partial response, or a relapse following a prolonged period of remission, subsequent courses of concurrent therapy may be needed to achieve complete remission of the disease. Thus, subsequent to a period of time off from a first treatment period, which may have comprised a constant IL-2 dosing regimen or a two-level IL-2 dosing regimen in combination with weekly, twice-weekly, or thrice-weekly administration of an anti-CD52 antibody, a subject may receive one or more additional treatment periods comprising either constant or two-level IL-2 dosing regimens in combination with anti-CD52 antibody administration. Such a period of time off between treatment periods is referred to herein as a time period of discontinuance. It is recognized that the length of the time period of discontinuance is dependent upon the degree of tumor response (i.e., complete versus partial) achieved with any prior treatment periods of concurrent therapy with these two therapeutic agents. Accordingly, the administration methods of the present invention provide an improved means for managing CLL in a human subject.

Thus, for example, where a subject is undergoing concurrent therapy with weekly, twice-weekly, or thrice-weekly doses of an anti-CD52 antibody and a constant IL-2 dosing regimen or a two-level IL-2 dosing regimen (which may or may not include a drug holiday between the first and second time periods of the two-level IL-2 dosing regimen), their treatment regimen may include multiple treatment sessions, each of which comprises concurrent therapy with weekly, twice-weekly, or thrice-weekly doses of an anti-CD52 antibody and a constant or two-level IL-2 dosing regimen. Monitoring of NK-cell count and T-cell count in a manner similar to that used to determine when a drug holiday must be concluded can be utilized to determine the necessity for multiple maintenance cycles. Thus, upon completion of any given period of concurrent therapy with an anti-CD52 antibody and a constant IL-2 dosing regimen or a two-level IL-2 dosing regimen, the treating physician obtains a measurement of NK-cell count and T-cell count. These indicators are then measured at monthly intervals (i.e., once a month) following completion of any given treatment period.

When a single measurement indicates that T-cell count has fallen below about 80% of that achieved at completion of the prior treatment period with concurrent IL-2/anti-CD52 antibody administration, a second measurement is made within about 1 week to about 2 weeks to confirm depletion of T-cell count below this value. If the second measurement also indicates a T-cell count less than about 80% of that achieved with the prior treatment period, this would be indicative of the need for administering another cycle of IL-2 dosing in combination with anti-CD52 antibody administration to the subject. In the situation wherein the CD4+ counts do not recover quickly enough, while the malignant cells recover earlier, this would be an indication for a repeated course of combination treatment as long as the T cell count is not too low. The duration between cycles of administration of these two therapeutic agents can be about 1 month to about 6 months, including 1 month, 1.5 months, 2 months, 2.5 months, 3 months, 3.5 months, 4 months, 4.5 months, 5 months, 5.5 months, 6 months, or other such time periods falling within the range of about 1 month to about 6 months.

IL-2 dosing schedule. For human subjects undergoing concurrent therapy with an anti-CD52 antibody according to the administration schedule recommended herein in combination with one or more cycles of a constant IL-2 dosing regimen or one or more cycles of a two-level IL-2 dosing regimen, the total weekly dose of an IL-2 to be administered during periods of IL-2 dosing can be administered as a single dose, or can be partitioned into a series of equivalent doses that are administered according to a two- three-, four-, five-, six-, or seven-times-a-week dosing schedule. Where higher total weekly doses are to be administered during a first time period, and lower total weekly doses are to be administered during a second time period, it is not necessary that the total weekly dose be administered in the same manner over the course of both dosing periods. Thus, for example, the higher total weekly dose during the first time period of a two-level IL-2 dosing regimen can be administered as a single dose, or can be partitioned into a series of equivalent doses that are administered according to a two- three-, four-, five-, six-, or seven-times-a-week dosing schedule. Similarly, the lower total weekly dose during the second time period of a two-level IL-2 dosing regimen can be administered as a single dose, or can be partitioned into a series of equivalent doses that are administered according to a two-, three-, four-, five-, six-, or seven-times-a-week dosing schedule.

For purposes of the present invention, a “two-, three-, four-, five-, six-, or seven-times-a-week dosing schedule” is intended to mean that the total weekly dose is partitioned into two, three, four, five, six, or seven equivalent doses, respectively, which are administered to the subject over the course of a 7-day period, with no more than one equivalent dose being administered per 24-hour period. The series of equivalent doses can be administered on sequential days, or can be administered such that one or more days occur between any two consecutive doses, depending upon the total number of equivalent doses administered per week.

Two equivalent doses: IL-2. Where a series of two equivalent doses of an IL-2 are administered per week (i.e., over a 7-day period) and the first equivalent dose of that week is administered on day 1, the second equivalent dose of an IL-2 can be administered on day 2, 3, 4, 5, 6, or 7 of that week. In one embodiment, the total weekly dose of an IL-2 is partitioned into two equivalent doses that are administered to the subject within a 7-day period, allowing for a minimum of 72 hours between doses and a maximum of 96 hours between doses.

Three equivalent doses: IL-2. Where a series of three equivalent doses of an IL-2 are administered per week and the first equivalent dose of that week is administered on day 1, the second equivalent dose can be administered on day 2, 3, 4, 5, or 6 of that week, and the third equivalent dose can be administered on day 3, 4, 5, 6, or 7 of that week, so long as about 24 hours occur between administration of the second and third equivalent doses. In some embodiments, the total weekly dose of an IL-2 is partitioned into three equivalent doses that are administered to the subject within a 7-day period, allowing for a minimum of 25 hours between doses and a maximum of 72 hours between doses. In other embodiments, the total weekly dose of an IL-2 is partitioned into three equivalent doses that are administered on day 1, day 3, and day 5 of each week of IL-2 administration. As noted previously, where an anti-CD52 antibody is also administered on day 1, day 3, and day 5 of each week of anti-CD52 antibody administration, these two agents can be administered at the same time, or sequentially, in either order.

Four equivalent doses: IL-2. Where a series of four equivalent doses of an IL-2 are administered per week and the first equivalent dose of that week is administered on day 1, the second equivalent dose can be administered on day 2, 3, 4, or 5 of that week, the third equivalent dose can be administered on day 3, 4, 5, or 6 of that week, and the fourth equivalent dose can be administered on day 4, 5, 6, or 7 of that week, so long as about 24 hours occur between administration of any two consecutive doses (i.e., between the first and second equivalent doses, between the second and third equivalent doses, and between the third and fourth equivalent doses).

Five equivalent doses: IL-2. Where a series of five equivalent doses of an IL-2 are administered per week and the first equivalent dose of that week is administered on day 1, the second equivalent dose can be administered on day 2, 3, or 4 of that week, the third equivalent dose can be administered on day 3, 4, or 5 of that week, the fourth equivalent dose can be administered on day 4, 5, or 6 of that week, and the fifth equivalent dose can be administered on day 5, 6, or 7 of that week, so long as about 24 hours occur between administration of any two consecutive doses (i.e., between the first and second equivalent doses, between the second and third equivalent doses, between the third and fourth equivalent doses, and between the fourth and fifth equivalent doses).

Six equivalent doses: IL-2. Where a series of six equivalent doses of an IL-2 are administered per week and the first equivalent dose of that week is administered on day 1, the second equivalent dose can be administered on day 2 or 3 of that week, the third equivalent dose can be administered on day 3 or 4 of that week, the fourth equivalent dose can be administered on day 4 or 5 of that week, the fifth equivalent dose can be administered on day 5 or 6 of that week, and the sixth equivalent dose can be administered on day 6 or 7 of that week, so long as about 24 hours occur between administration of any two consecutive doses (i.e., between the first and second equivalent doses, between the second and third equivalent doses, between the third and fourth equivalent doses, between the fourth and fifth equivalent doses, and between the fifth and sixth equivalent doses).

Seven equivalent doses: IL-2. In one embodiment, the total weekly dose of an IL-2 is partitioned into seven equivalent doses, which are administered daily over the 7-day period, with about 24 hours occurring between each consecutive dose.

Adjustment of dosing schedule: IL-2. It is not necessary that the same dosing schedule be followed throughout a constant IL-2 dosing regimen, or that the same dosing schedule be followed for both the first and second periods of the two-level IL-2 dosing regimen. Thus, the dosing schedule can be adjusted to accommodate an individual's tolerance of prolonged IL-2 therapy in combination with anti-CD52 antibody therapy, and to reflect the individual's responsiveness to concurrent therapy with these two therapeutic agents. The preferred dosing schedule during the constant IL-2 dosing regimen and the two time periods of the two-level IL-2 dosing regimen is readily determined by the managing physician given the patient's medical history and the guidance provided herein.

Anti-CD52 antibody dose ranges. The present invention provides methods for treating a human subject with chronic lymphocytic leukemia, and other cancers whose unabated proliferating cells express the CD52 cell-surface antigen, using concurrent therapy with an anti-CD52 antibody administered according to the dosing schedule recommended herein in combination with one or more cycles of either a constant IL-2 dosing regimen or a two-level IL-2 dosing regimen. For purposes of the present invention, the therapeutically effective dose of an anti-CD52 antibody to be administered on a weekly, twice-weekly, or thrice-weekly dosing schedule ranges from about 10 mg to about 30 mg, including about 10 mg, 12 mg, 15 mg, 17 mg, 20 mg, 22 mg, 25 mg, 27 mg, 30 mg, and other such doses falling within the range of about 10 mg to about 30 mg. The same therapeutically effective dose of an anti-CD52 antibody can be administered throughout each week of dosing. Alternatively, different therapeutically effective doses of an anti-CD52 antibody can be used over the course of a treatment period.

Thus, in some embodiments, the initial therapeutically effective dose of an anti-CD52 antibody can be in the lower dosing range (i.e., about 3 mg to about 10 mg), with subsequent doses falling within the higher dosing range (i.e., to about 30 mg). In one embodiment, the subject is administered an initial dose of about 3 or 10 mg of an anti-CD52 antibody on day 1 of a treatment period, and is then administered escalating doses of the antibody until a dose of about 30 mg is reached, preferably within about 1 to 2 weeks following initiation of anti-CD52 antibody administration. In one such embodiment, the first dose of 3 or 10 mg of an anti-CD52 antibody is administered on day 1 of the treatment period, about 10 or 30 mg of an anti-CD52 antibody is administered on day 3 of that treatment period, about 30 mg of an anti-CD52 antibody is administered on day 5 of that treatment period, and then all subsequent doses of an anti-CD52 antibody are administered at a dose of about 30 mg. Thus, such a subject would then be administered 30 mg of an anti-CD52 antibody according to a weekly, twice-weekly, or thrice-weekly dosing schedule starting on day 1 of the second week of anti-CD52 antibody administration (i.e., on day 8 of the treatment period). In such embodiments, it is preferable to begin IL-2 administration on day 8 of the treatment period. The pharmaceutical composition comprising an anti-CD52 antibody is administered, for example, subcutaneously or intravenously, as noted herein above.

Routes of administration. In accordance with the methods of the present invention, an IL-2 is administered preferably by SC injection, in combination with an anti-CD52 antibody therapy so as to provide the recommended total weekly doses of IL-2 during the constant IL-2 dosing regimen or during the two-level IL-2 dosing regimen as described more fully below. The following embodiments provide guidance as to suitable total weekly doses and dosing regimens for IL-2, though any number of different dosing regimens can be contemplated by one of skill in the art once apprised of the disclosure set forth herein.

Reference IL-2 standard. For purposes of the following discussion, the multimeric IL-2 pharmaceutical composition commercially available under the trade name Proleukin® IL-2 (in some markets, Proleukin®) (Chiron Corporation, Emeryville, Calif.) is used as the reference IL-2 standard. By “reference IL-2 standard” is intended the formulation of IL-2 that serves as the basis for determination of the total weekly IL-2 doses to be administered to a human subject with CLL in accordance with the desired dosing regimen in combination with at least one anti-CD52 antibody to achieve the desired positive effect, i.e., a T-cell protective effect and potentially also a positive therapeutic response that is improved with respect to that observed with an anti-CD52 antibody alone.

Where Proleukin® IL-2 is to be administered according to a constant IL-2 dosing regimen, the total weekly dose is about 18.0 MIU to about 42.0 MIU, while the therapeutically effective dose of anti-CD52 antibody to be administered weekly, twice-weekly, or thrice-weekly is usually in the range from about 10 mg to about 30 mg as noted above although up to 80 mg can be used. Thus, for example, in some embodiments, the total amount of Proleukin® IL-2 that is to be administered per week as part of a constant IL-2 dosing regimen is about 18.0 MIU, 20.0 MIU, 22.0 MIU, 24.0 MIU, 26.0 MIU, 28.0 MIU, 30.0 MIU, 32.0 MIU, 34.0 MIU, 36.0 MIU, 38.0 MIU, 40.0 MIU, or 42.0 MIU, and other such values falling in the range of 18.0 MIU and 42.0 MIU, and the therapeutically effective dose of anti-CD52 antibody is about 10, 12, 15, 17, 20, 22, 25, 27, or 30 mg, or other such values falling within the range of about 10 mg to about 30 mg, dosed weekly, twice-weekly, or thrice-weekly. In one embodiment, the total weekly dose of Proleukin® IL-2 is about 42.0 MIU, and the total amount of anti-CD52 antibody is about 30 mg dose. As previously noted, the total weekly dose of IL-2 during the first and second time periods of a two-level IL-2 dosing regimen is administered as a single dose, or is partitioned into a series of equivalent doses that are administered according to a two-, three-, four-, five-, six-, or seven-times-a-week dosing schedule.

Thus, for example, where the total weekly dose of Proleukin® IL-2 during the constant IL-2 dosing regimen is 42.0 MIU, the three equivalent doses of this reference IL-2 standard to be administered during each week would be 14.0 MIU, and the two equivalent doses of this reference IL-2 standard to be administered during each week would be 21.0 MIU. Similarly, where the total weekly dose of Proleukin® IL-2 during the constant IL-2 dosing regimen is 18.0 MIU, the three equivalent doses of this reference IL-2 standard to be administered during each week would be 6.0 MIU, and the two equivalent doses of this reference IL-2 standard to be administered during each week would be 9.0 MIU.

Where Proleukin® IL-2 is to be administered according to a two-level IL-2 dosing regimen, the higher total weekly dose that is administered during the first time period of this dosing regimen is about 18.0 MIU to about 42.0 MIU, and the lower total weekly dose that is administered during the second time period of this dosing regimen is about 18.0 MIU to about 30.0 MIU. As previously noted, the total weekly dose administered during the first time period of the two-level IL-2 dosing regimen, for example, during the first half of this dosing regimen, is always higher than the total weekly dose administered during the second time period of the two-level IL-2 dosing regimen, for example, during the second half of this dosing regimen.

Thus, in some embodiments, the higher total weekly dose of Proleukin® IL-2 that is administered during the first time period of the two-level IL-2 dosing regimen is about 18.0 MIU to about 42.0 MIU, including about 18.0 MIU, 20.0 MIU, 22.0 MIU, 24.0 MIU, 26.0 MI, 28.0 MIU, 30.0 MIU, 32.0 MIU, 34.0 MI, 36.0 MIU, 38.0 MIU, 40.0 MIU, or 42.0 MIU, or other such values falling within this higher dosing range; and the lower total weekly dose of Proleukin® IL-2 is about 18.0 MIU to about 30.0 MIU, including 18.0 MIU, 20.0 MIU, 22.0 MIU, 24.0 MIU, 26.0 MIU, 28.0 MIU, or 30.0 MIU, or other such values falling within this lower dosing range. In one embodiment, the two-level IL-2 dosing regimen has a combined duration of 10 weeks to 20 weeks, where the higher total weekly dose of Proleukin® IL-2 that is administered during the first time period of the two-level IL-2 dosing regimen is about 38.0 MIU to about 42.0 MIU, such as 38.0 MIU, 39.0 MIU, 40.0 MIU, 41.0 MIU, or 42.0 MIU, and the lower total weekly dose of Proleukin® IL-2 that is administered during the second time period of the two-level IL-2 dosing regimen is about 18.0 MIU to about 30.0 MIU, such as 18.0, 20.0, 22.0, 24.0, 26.0, 28.0, or 30.0 MIU. In one such embodiment, the higher total weekly dose of Proleukin® IL-2 that is administered during the first time period is 42.0 MIU and the lower total weekly dose of Proleukin® IL-2 that is administered during the second time period is 30.0 MIU.

As previously noted, the total weekly dose of IL-2 during the first and second time periods of a two-level IL-2 dosing regimen is administered as a single dose, or is partitioned into a series of equivalent doses that are administered according to a two-, three-, four-, five-, six-, or seven-times-a-week dosing schedule. Thus, for example, where the total weekly dose of Proleukin® IL-2 during the first period of the two-level IL-2 dosing regimen is 42.0 MIU, the three equivalent doses of this reference IL-2 standard to be administered during each week would be 14.0 MIU, and the two equivalent doses of this reference IL-2 standard to be administered during each week would be 21.0 MIU. Similarly, where the total weekly dose of Proleukin® IL-2 during the second period of the two-level IL-2 dosing regimen is 30.0 MIU, the three equivalent doses of this reference IL-2 standard to be administered during each week would be 10.0 MIU, and the two equivalent doses of this reference IL-2 standard to be administered during each week would be 15.0 MIU.

The foregoing therapeutically effective doses of the reference IL-2 standard Proleukin® IL-2 are expressed in terms of MIU, which represent total amounts or absolute doses that are to be administered to a human subject on a weekly basis. The corresponding relative total weekly dose of Proleukin® IL-2 to be administered to a person can readily be calculated. The average person is approximately 1.7 m². Thus, where the absolute total weekly dose of Proleukin® IL-2 to be administered is about 30.0 MIU to about 42.0 MIU, the corresponding relative total weekly dose of Proleukin® IL-2 is about 17.6 MIU/m² to about 24.7 MIU/m2. Similarly, when the absolute total weekly dose is 30.0 MIU, 32.0 MIU, 35.0 MIU, 37.0 MIU, 40.0 MIU, or 42.0 MIU, the corresponding relative total weekly dose is 17.6 MIU/m², 18.8 MIU/m², 20.6 MIU/m², 21.8 MIU/m², 23.5 MIU/m², and 24.7 MIU/m², respectively. Where the absolute total weekly doses that are to be administered are within the range of about 18.0 MIU to about 30.0 MIU, including, for example, 18.0 MIU, 20.0 MIU, 23.0 MIU, 25.0 MIU, 27.0 MIU, 30.0 MIU, the corresponding relative total weekly doses are about 10.6 MIU/m² to about 17.6 MIU/m², including 10.6 MIU/m², 11.8 MIU/m², 13.5 MIU/m², 14.7 MIU/m², 15.9 MIU/m², and 17.6 MIU/m², respectively.

Calculation of IL-2 protein doses for aldesleukin. This section relates to calculating doses for aldesleukin and variants thereof, including embodiments in which the administration is SC, IV or IM. MIU (“million international units”) represents an international convention for describing a protein's biological activity. The international unit for IL-2 biological activity was established in 1988 by the World Health Organization (WHO) International Laboratory for Biological Standards. The IL-2 biological reference materials provided by the National Institute for Biological Standards and Control (NIBSC), which belongs to WHO, has 100 international units per ampoule of native human, Jurkat-derived IL-2. Activity of an IL-2 product can be measured against this international standard in an in vitro potency assay by HT-2 cell proliferation. Thus, for example, Proleukin® IL-2 has a biological activity of about 16.36 MIU per mg of this IL-2 product as determined by an HT-2 cell proliferation assay (see, for example, Gearing and Thorpe (1988) J. Immunological Methods 114:3-9; Nakanishi et al. (1984) J. Exp. Med. 160(6):1605-1621). The active moiety used in this product is the recombinant human IL-2 mutein aldesleukin (referred to as des-alanyl-1, serine-125 human interleukin-2; see U.S. Pat. No. 4,931,543). Using this information, one can calculate the recommended absolute total weekly dose of Proleukin® IL-2 in micrograms. Hence, where the absolute total weekly dose of Proleukin® IL-2 is about 30.0 MIU to about 42.0 MIU, the corresponding absolute total weekly dose of Proleukin® IL-2 in micrograms is about 1833 μg to about 2567 μg of this product. Similarly, where the absolute total weekly dose in MIU is about 18.0 MIU to about 30.0 MIU, the corresponding absolute total weekly dose in μg is about 1100 μg to about 2567 μg. Thus, given an absolute total weekly dose of Proleukin® IL-2 expressed in MIU, one of skill in the art can readily compute the corresponding relative total weekly dose expressed in MIU/m2, or the absolute total weekly dose expressed in μg of this IL-2 product.

For purposes of describing this invention, the doses of IL-2 have been presented using Proleukin® IL-2 as the reference IL-2 standard. One of skill in the art can readily determine what the corresponding doses would be for different aldesleukin formulations and routes of administration using a conversion factor based on comparative pharmacokinetic (PK) data and the serum concentration-time curve (AUC) for PK data collected during a 24-hour period for Proleukin® IL-2. Using PK data, the IL-2 exposure in human subjects that were administered a single dose of the reference IL-2 standard was determined. These subjects were selected such that they had not previously received exogenous IL-2 therapy (i.e., these subjects were naive to IL-2 therapy). By “exogenous IL-2 therapy” is intended any intervention whereby a subject has been exposed to an exogenous source of IL-2, as opposed to exposure that occurs with the body's production of naturally occurring IL-2. Some of these subjects had received a single dose of 4.5 MIU of the reference IL-2 standard, while others had received a single dose of 7.5 or 18.0 MIU of the reference IL-2 standard.

Following administration of the single dose of the reference IL-2 standard, the IL-2 exposure in the blood serum was monitored over the first 10 to 12 hours post injection, then extrapolated to 24 hours, and the resulting area under the serum concentration-time curve (AUC) for data collected during that 24-hour period was calculated. This area under the serum concentration-time curve is referred to herein as the AUC₀₋₂₄. Methods for measuring IL-2 exposure in this manner are well known in the art. See, for example, Gustavson (1998) J. Bio. Response Modifiers 1998:440-449; Thompson et al. (1987) Cancer Research 47:4202-4207; Kirchner et al. (1998) Br. J. Clin. Pharmacol. 46:5-10; Piscitelli et al. (1996) Pharmacotherapy 16(5):754-759; and Example 3 below. Thus, for those subjects receiving a dose of 4.5 MIU (300 μg) of Proleukin® IL-2, the AUC₀₋₂₄ value was 51 IU*hr/ml (SD=14); for those subjects receiving a dose of 7.5 MIU (458 μg) of Proleukin® IL-2, the AUC₀₋₂₄ value was 79 IU*hr/ml (SD=29); and for those subjects receiving the 18.0 MIU dose of Proleukin® IL-2 (1100 μg), the AUC₀₋₂₄ value was 344 IU*hr/ml (SD=127). When such AUC₀₋₂₄ data is determined for the reference IL-2 standard, Proleukin® IL-2, the therapeutically effective doses described herein result in an IL-2 exposure within a range from about 23 IU*hr/ml serum to about 598 IU*hour/ml serum.

The sum of individual AUC₀₋₂₄ from individual doses will comprise the total weekly AUC₀₋₂₄ in partitioned individual doses. For example, if a dose of 18 MIU is administered three-times-a-week, the individual AUC₀₋₂₄ is estimated at 375 IU*hr/ml, and the total weekly AUC₀₋₂₄ will be 1132 IU*hr/ml based on linear assumption of increased AUC₀₋₂₄ with dose as shown in the Table 1 below.

TABLE 1
AUC₀₋₂₄ values obtained after administration of Proleukin ® IL-2

| Proleukin ® IL-2 Dose | AUC₀₋₂₄ |
|---|---|
| AUC₀₋₂₄ (MIU/μg) | (IU * hr/ml) |
| 18/1100 | 375 |
| 30/2000 | 625 |
| 42/2800 | 875 |
| 54/3600 | 1032 |

The same total weekly AUC₀₋₂₄ of 1032 IU*hr/ml could also be obtained by dosing two-times-a-week at 27 MIU or dosing five-times-a-week at about 11 MIU.

Calculation of protein doses other than aldesleukin. Variants of IL-2 may have altered intrinsic biological activity. To estimate the dose of an IL-2 variant other than aldesleukin that will be comparable to the doses of aldesleukin disclosed herein, the relative biological activity of the IL-2 variant should be determined by testing its effect.

Such effects include NK cell activity expansion and ability to activate and/or expand normal T cells (CD or CD8). Therefore, relevant biological activity of an IL-2 according to the invention is the ability to achieve expansion of human NK cells. Such activity is measured using a standard assay such as that disclosed in Nagler, P., et al. (1989) J. Immunol. 143:3183-3191. To determine suitable doses of IL-2 variants that are not aldesleukin, discussed above, a series of doses of an IL-2 is administered to human subjects and freshly isolated PBMC (peripheral blood mononuclear cells) from the subjects are tested for NK cytotoxicity against K562 cells as described, for example, by Nagler, et al., as described above. The NK stimulatory effect of the IL-2 being tested is compared with that of an IL-2 standard as described above when administered in the same μg amounts by the same route of administration. The IL-2 variant will have an NK cell stimulatory potency that is preferably 100% that of the same amount of a reference IL-2 standard, or 90%, 80%, 70%, or 60% that of the same amount of a reference IL-2 standard. The IL-2 variant should be substantially biologically active, as defined as having at least 50% of the NK stimulatory activity of the same dose of a reference IL-2 standard, administered by the same route.

Pharmaceutical compositions. Any pharmaceutical composition comprising an IL-2 as the therapeutically active component can be used in the methods of the invention. Such pharmaceutical compositions are known in the art and include, but are not limited to, those disclosed in U.S. Pat. Nos. 4,745,180; 4,766,106; 4,816,440; 4,894,226; 4,931,544; and 5,078,997. Thus liquid, lyophilized, or spray-dried compositions comprising an IL-2 that are known in the art may be prepared as an aqueous or nonaqueous solution or suspension for subsequent administration to a subject in accordance with the methods of the invention. Each of these compositions will comprise an IL-2 as a therapeutically or prophylactically active component. By “therapeutically or prophylactically active component” is intended an IL-2 is specifically incorporated into the composition to bring about a desired therapeutic or prophylactic response with regard to treatment, prevention, or diagnosis of a disease or condition within a subject when the pharmaceutical composition is administered to that subject. Preferably the pharmaceutical compositions comprise appropriate stabilizing agents, bulking agents, or both to minimize problems associated with loss of protein stability and biological activity during preparation and storage.

In preferred embodiments of the invention, IL-2 containing pharmaceutical compositions useful in the methods of the invention are compositions comprising stabilized monomeric IL-2, compositions comprising multimeric IL-2, and compositions comprising stabilized lyophilized or spray-dried IL-2.

As the invention also provides for the use of an IL-2 or an anti-CD52 antibody in the manufacture of a medicament for use in treatment of CLL in an individual any IL-2 as defined herein is suitable for such a use. By the term “medicament” is meant a composition of manufacture with at least two components, an IL-2 and an anti-CD52 antibody. The two components are maintained separately prior to administration. In some embodiments, the anti-CD52 antibody is Alemtuzumab.

The invention also provides a kit comprising an anti-CD52 antibody, and instructions for delivering the anti-CD52 antibody to an individual suffering from CLL, prior to or with delivering an IL-2, wherein the instructions provide for one cycle of concurrent therapy with the anti-CD52 antibody and the IL-2. In some embodiments of the kit, the anti-CD52 antibody is Alemtuzumab. Where more than one cycle of concurrent therapy is desired, more than one kit is employed. In some embodiments, components of the kit which provide for one cycle can be combined, for producing a kit having adequate amounts of an anti-CD52 antibody and instructions for administering more than one cycle. In specific embodiments, the anti-CD52 antibody is packaged to provide for the various dosing regimens described herein.

Also provided is a kit comprising an IL-2, and instructions for delivering the IL-2 to an individual suffering from CLL, with concurrent therapy using an anti-CD52 antibody, wherein the instructions provide for one cycle of concurrent therapy with the IL-2 and the anti-CD52 antibody. Where more than one cycle of concurrent therapy is desired, more than one kit is employed. In some embodiments, components of the kit which provide for one cycle can be combined, for producing a kit having adequate amounts of an IL-2 and instructions for administering more than one cycle. In specific embodiments, the IL-2 is packaged to provide for the various dosing regimens described herein.

The invention also provides for use of an interleukin-2 (IL-2) in the preparation of a medicament for treating chronic lymphocytic leukemia (CLL) in a human subject previously administered with, or receiving administration of, an anti-CD52 antibody; use of an anti-CD52 antibody in the preparation of a medicament for treating CLL in a human subject previously administered with, or receiving administration of, an IL-2; use of an IL-2 in the preparation of a medicament for treating CLL in a human subject by separate, sequential or simultaneous administration with an anti-CD52 antibody; and use of an anti-CD52 antibody in the preparation of a medicament for treating CLL in a human subject by separate, sequential or simultaneous administration with an IL-2. The invention further provides for use of an anti-CD52 antibody and an IL-2 in the preparation of a medicament for treating CLL in a human subject by separate, sequential or simultaneous administration of the anti-CD52 antibody and the IL-2; and use of an IL-2 in the preparation of a medicament for increasing or maintaining T-cell count in a human subject previously administered with, or receiving administration of, an anti-CD52 antibody. In some embodiments, the anti-CD52 antibody is administered to the subject according to a weekly, twice-weekly or thrice-weekly dosing schedule; in other embodiments, the IL-2 is administered to the subject according to a constant dosing regimen comprising administering a total weekly dose of an IL-2 to the subject.

A first dose of the IL-2 may be administered to the subject simultaneously with a first dose of the anti-CD52 antibody, and a first dose of the IL-2 may be administered to the subject one week after a first dose of the anti-CD52 antibody. In certain embodiments of these uses, the total weekly dose of the IL-2 is in an amount that provides at least 50% of the NK stimulatory activity of a total weekly dose of Aldesleukin administered in a range of from about 1100 μg to about 1834 μg.

The invention also provides a kit comprising an anti-CD52 antibody, an IL-2 and instructions for administering the IL-2, separately, simultaneously or sequentially with administration of the anti-CD52 antibody, to an individual suffering from CLL; in some embodiments, the instructions are to administer the IL-2 following the administration of the anti-CD52 antibody.

In embodiments of the use or kit IL-2 is recombinantly produced IL-2 having an amino acid sequence for human IL-2 or a variant thereof having at least 70% sequence identity to the amino acid sequence for human IL-2; the variant may be des-alanyl-1, serine 125 human interleukin-2.

In the embodiments of the use or kit the anti-CD52 antibody is an immunologically active anti-CD52 antibody, and the anti-CD52 antibody may be Alemtuzumab or a fragment thereof.

In further embodiments of the use or kit the anti-CD52 antibody is a human anti-CD52 antibody, a humanized anti-CD52 antibody, or a chimeric anti-CD52 antibody.

Monomeric IL-2 compositions. By “monomeric” IL-2 is intended the protein molecules are present substantially in their monomer form, not in an aggregated form, in the pharmaceutical compositions described herein. Hence covalent or hydrophobic oligomers or aggregates of IL-2 are not present. Such compositions are contemplated for use as “an IL-2” according to this invention. Exemplary pharmaceutical compositions comprising stabilized monomeric IL-2 are disclosed in International Publication No. WO 01/24814, entitled “Stabilized Liquid Polypeptide-Containing Pharmaceutical Compositions,” incorporated herein by reference.

These liquid pharmaceutical compositions comprising stabilized monomeric IL-2 may either be used in an aqueous liquid form, or stored for later use in a frozen state, or in a dried form for later reconstitution into a liquid form or other form suitable for administration to a subject in accordance with the methods of present invention. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essex, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53).

Other examples of IL12 formulations that comprise an IL-2 in its nonaggregated monomeric state include those described in Whittington and Faulds (1993) Drugs 46(3):446-514. These formulations include the recombinant IL-2 product in which the recombinant IL-2 mutein Teceleukin (unglycosylated human IL-2 with a methionine residue added at the amino-terminal) is formulated with 0.25% human serum albumin in a lyophilized powder that is reconstituted in isotonic saline, and the recombinant IL-2 mutein Bioleukin (human IL-2 with a methionine residue added at the amino-terminal, and a substitution of the cysteine residue at position 125 of the human IL-2 sequence with alanine) formulated such that 0.1 to 1.0 mg/ml IL-2 mutein is combined with acid, wherein the formulation has a pH of 3.0 to 4.0, advantageously no buffer, and a conductivity of less than 1000 mmhos/cm (advantageously less than 500 mmhos/cm). See EP 373,679; Xhang et al. (1996) Pharmaceut. Res. 13(4):643644; and Prestrelski et al. (1995) Pharmaceut. Res. 12(9): 1250-1258.

Multimeric IL-2 compositions. Examples of pharmaceutical compositions comprising multimeric IL-2 are disclosed in commonly owned U.S. Pat. No. 4,604,377. By “multimeric” is intended the protein molecules are present in the pharmaceutical composition in a microaggregated form having an average molecular association of 10-50 molecules. These multimers are present as loosely bound, physically associated IL-2 molecules. A lyophilized form of these compositions is available commercially under the trade name Proleukin® IL-2 (Chiron Corporation). The lyophilized formulations disclosed in this reference comprise selectively oxidized, microbially produced recombinant IL-2 in which the recombinant IL-2 is admixed with a water soluble carrier such as mannitol that provides bulk, and a sufficient amount of sodium dodecyl sulfate to ensure the solubility of the recombinant IL-2 in water. These compositions are suitable for reconstitution in aqueous injections for parenteral administration and are stable and well tolerated in human patients. When reconstituted, the IL-2 retains its multimeric state. Such lyophilized or liquid compositions comprising multimeric IL-2 are encompassed by the methods of the present invention. Such compositions are referred to herein as multimeric IL-2 pharmaceutical compositions.

Pharmaceutical composition. The methods of the present invention may also use stabilized lyophilized or spray-dried pharmaceutical compositions comprising IL-2, which may be reconstituted into a liquid or other suitable form for administration in accordance with methods of the invention. Such pharmaceutical compositions are disclosed in International Publication No. WO 01/24814, entitled “Methods for Pulmonary Delivery of Interleukin-2.” These compositions may further comprise at least one bulking agent, at least one agent in an amount sufficient to stabilize the protein during the drying process, or both. By “stabilized” is intended an IL-2 protein that retains its monomeric or multimeric form as well as its other key properties of quality, purity, and potency following lyophilization or spray-drying to obtain the solid or dry powder form of the composition. In these compositions, preferred carrier materials for use as a bulking agent include glycine, mannitol, alanine, valine, or any combination thereof, most preferably glycine. The bulking agent is present—in the formulation in the range of 0% to about 10% (w/v), depending upon the agent used. Preferred carrier materials for use as a stabilizing agent include any sugar or sugar alcohol or any amino acid. Preferred sugars include sucrose, trehalose, raffinose, stachyose, sorbitol, glucose, lactose, dextrose or any combination thereof, preferably sucrose. When the stabilizing agent is a sugar, it is present in the range of about 0% to about 9.0% (w/v), preferably about 0.5% to about 5.0%, more preferably about 1.0% to about 3.0%, most preferably about 1.0%. When the stabilizing agent is an amino acid, it is present in the range of about 0% to about 1.0% (w/v), preferably about 0.3% to about 0.7%, most preferably about 0.5%. These stabilized lyophilized or spray-dried compositions may optionally comprise methionine, ethylenediaminetetracetic acid (EDTA) or one of its salts such as disodium EDTA or other chelating agent, which protect the IL-2 against methionine oxidation. Use of these agents in this manner is described in International Publication No. WO 01/24814. The stabilized lyophilized or spray-dried compositions may be formulated using a buffering agent, which maintains the pH of the pharmaceutical composition within an acceptable range, preferably between about pH 4.0 to about pH 8.5, when in a liquid phase, such as during the formulation process or following reconstitution of the dried form of the composition. Buffers are chosen such that they are compatible with the drying process and do not affect the quality, purity, potency, and stability of the protein during processing and upon storage.

The previously described stabilized monomeric, multimeric, and stabilized lyophilized or spray-dried IL-2 pharmaceutical compositions represent suitable compositions for use in the methods of the invention. However, any pharmaceutical composition comprising an IL-2 as a therapeutically active component is encompassed by the methods of the invention.

### EXAMPLE 1

### Combination Treatment with an Anti-CD52 Antibody and an IL-2

This example describes an open label, uncontrolled trial. All subjects receive escalating doses (e.g., 3 mg-10 mg-30 mg on days 1, 3 and 5) of Alemtuzumab subcutaneously (SC) during week 1 until the dose of 30 mg SC is reached and well tolerated. If the intended escalation is not tolerated within 1 week, the escalation should be done within 2 weeks and all subsequent study procedures will also be delayed by one week.

Treatment with an IL-2 starts preferably on day 8 after the dose of 30 mg Alemtuzumab is reached. Subjects receive 3 weekly SC injections of IL-2 on the same days as Alemtuzumab for 11 weeks and then continue to receive IL-2 at the same dose for another 4 weeks after finishing the treatment with Alemtuzumab.

The first six subjects receive IL-2 at a dose of 10 MIU SC three times weekly for total 15 weeks. In case of dose-limiting toxicity (DLT) in 2 or more out of 6 patients the dose of IL-2 will be reduced to 6 MIU three times weekly. If the dosing is tolerated without dose limiting toxicity, the dose will be increased in another 6 subjects to 14 MIU three times weekly.

If the dose of 14 MIU is tolerated well and no DLT is observed, more patients will be included at the same dose level up to a total number of 25. If 14 MIU three times weekly is not tolerated well, patients will be included at the previous dose level.

Safety will be evaluated based on CTC criteria, including clinical and laboratory changes. Efficacy will be evaluated based on the number of complete and partial responses (NCI Working Group, Cheson, B., et al., 1996) and on percent change of T cells and NK cells in the peripheral blood. T cell and NK cell and NK-T cell numbers will be correlated with IL-2 and IL-2R plasma levels. The pharmacokinetics of IL-2 will be evaluated in selected patients.

The following criteria apply to this study:

Number of subjects: 25

Diagnosis and main criteria for inclusion and exclusion:

Inclusion Criteria:
Patients with B-cell chronic lymphocytic leukemia, relapsed or refractory following treatment with chemotherapy including purine analogue agents (e.g., fludarabine, cladribine)
Confirmed diagnosis of B CLL
CD52 positive disease
Age≧18 years
Written informed consent
ECOG performance status≦1
Patient requires treatment for CLL (Rai stage III or IV or Rai stage I-II with at least one of the following: evidence of progressive bone marrow involvement [manifested by development or worsening of anemia and/or thrombocytopenia], massive (>6 cm below left costal margin) or progressive splenomegaly, progressive lymphocytosis with 50% increase over a 2 months period or anticipated doubling time of less than 6 months, lymphocyte count<100,000/mm3, presence of B symptoms.

Exclusion Criteria:
Autoimmune anemia and/or thrombocytopenia
Any other active autoimmune disease
Prior treatment with Campath® or IL-2
Transformation into high grade lymphoma
Clinically significant pulmonary dysfunction
Clinically significant cardiac dysfunction or history of myocardial infarct or uncompensated congestive heart failure within 6 months before study (NYHA>2)
Chronic infections (e.g., HCV)

Other standard exclusion criteria also apply. The inclusion and exclusion criteria are provided as guidelines and should not preclude inclusion or exclusion of specific patients or patient groups who do not meet the specific criteria but nonetheless are appropriate for treating or not treating in the view of the physician.

Proleukin® (Aldesleukin) IL-2, hereafter referred to as IL-2, is administered SC. The dose is initially 10 MIU three times/week for 15 weeks. If tolerated in 6 subjects, the dose is increased to 14 MIU three times/week for 15 weeks.

Alemtuzumab is administered as SC injection, 3× weekly (days 1, 3 and 5). Gradually escalating doses will be given during week 1 until a daily dose of 30 mg is reached. Thereafter a dose of 30 mg is given SC, 3× weekly for another 11 weeks.

Duration of treatment:

Alemtuzumab—12 weeks

IL-2—15 weeks

The criteria for evaluation are as follows:

Efficacy

Objective response will be evaluated according to NCI Working Group criteria (Cheson B. et al., 1996) at weeks and 17. CR and PR must be confirmed by a repeated analysis 1-2 months later, preferably a CT scan 4 weeks later. After the end of the treatment period, CT scans and bone marrow examinations will be done at least every 3 months for another 18 months (approximately 2 years after start of study) or until progression (whichever is earlier).

Lymphocyte subsets are evaluated in the peripheral blood at baseline, weekly until week 4, and thereafter every 2 weeks until week 17. During follow-up FACS analysis will be done at least once monthly. The number of cells expressing CD5, CD19, CD20, CD25, CD4, CD8, CD16 and CD56, CD52 (incl. CD4+/CD52+, CD8+/CD52+ and CD19+/CD52+) will be evaluated using flow cytometry. Soluble IL-2R (sCD25) will be measured using standard (ELISA) procedure. The plasma levels of IL-2 will be measured in blood samples of selected patients.

Safety

Clinical signs and adverse events will be followed throughout the study and documented at least weekly. CTC criteria will be used to classify drug-related toxicity. Clinical laboratory evaluations will be carried out weekly, including chemistry, hematology and coagulation parameters. Immunogenicity (anti-IL-2, anti-Campath®) will be evaluated at baseline and at week 17.

Statistical Methods: Safety parameters and tumor responses will be tabulated. Response rates and safe data will be correlated with the increase of T cells and NK cells in a descriptive manner.

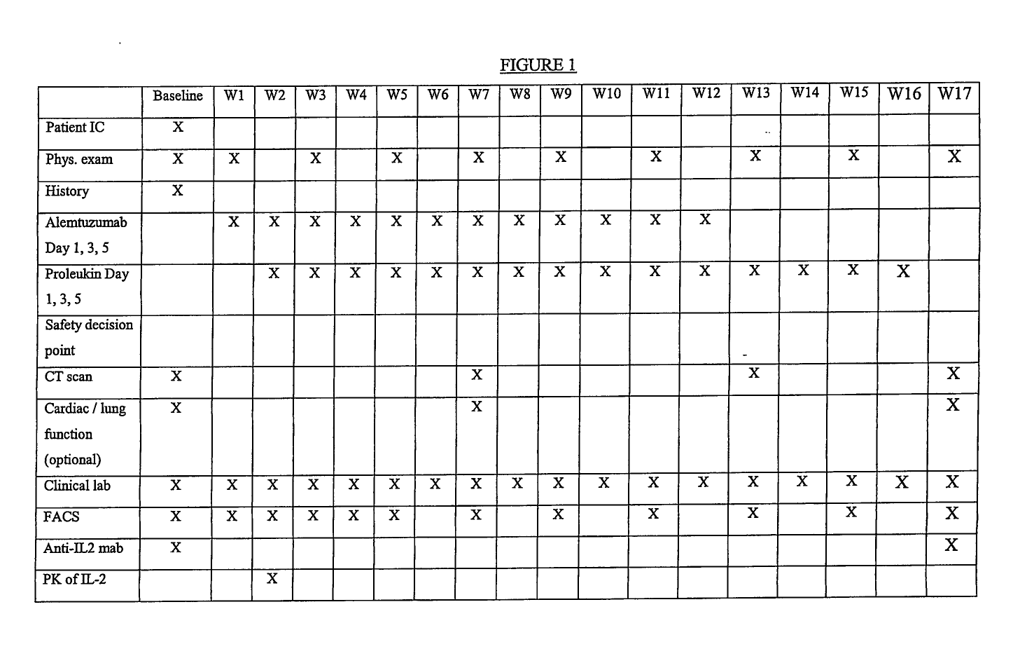

The schedule of events is summarized in FIG. 1.

The IL-2 formulation used in this study is manufactured by Chiron Corporation of Emeryville, Calif., under the trade name Proleukin® IL-2. The IL-2 in this formulation is a recombinantly produced, unglycosylated human IL-2 mutein, called aldesleukin, which differs from the native human IL-2 amino acid sequence in having the initial alanine residue eliminated and the cysteine residue at position 125 replaced by a serine residue (referred to as des-alanyl-1, serine-125 human interleukin-2). This IL-2 mutein is expressed in E. coli, and subsequently purified by diafiltration and cation exchange chromatography as described in U.S. Pat. No. 4,931,543. The IL-2 formulation marketed as Proleukin® IL-2 is supplied as a sterile, white to off-white preservative-free lyophilized powder in vials containing 1.3 mg of protein (22 MIU).

### EXAMPLE 2

### In Vivo Model Systems to Evaluate Campath® IH and IL-2

Campath®-1H is directed against the CD52 antigen, which is a 21-28 kDa MIU glycoprotein antigen that is coupled to the membrane by a glycophophatidylinositol (GPI) anchoring structure. CD52 is expressed on normal and neoplastic T and B-lymphocytes, natural killer (NK) cells, monocytes and monocyte-derived dendritic cells and macrophages, and tissues of the male reproductive system.

Human cell lines expressing CD52 include the B-cell line, Wien 133 Cl which expresses high levels of CD52, human non-Hodgkin's follicular B-cell lymphoma cell lines DoHH2 and BEVA carrying the t(14;18) translocation (Kluin-Nelemans et al. (1991) Leukemia S: 221; de Kroon et al., (1994). Leukemia 8: 1385-1391 and de Kroon et al. (1996) Exp. Hematol. 24: 919-926), human myeloid cell line expressing both FcR and CD52 molecules (THP-1; Lindmo et al., (1984) J. Immunol. Methods 72: 77-84; Fleit and Kobasiuk, (1991) J. Leukocyte Biol. 49: 556-565), CHO—IOID4 (Hutchins et al., (1995) Proc. Natl. Acad. Sci. USA; 92: 11980-11984) and Jurkat T cells transfected and selected to express high levels of CD52, as well as CD52 expressing cells from untreated, relapsed, or refractory (e.g fludarabine) chronic lymphocytic leukemia (B-CLL) and T-cell prolymphocytic leukemia patients (CLL Pro PLL; O'Brien et al., (1996) Leukemia 10: 338-344) or other CD52 expressing tumor types engrafted via subcutaneously, IP or IV routes into nude or SCID mice.

Campath® is believed to act predominantly via an antibody dependent cellular cytotoxicity (ADCC) mechanism although cell growth inhibition via Campath®-mediated CD52 signaling has been reported (Rowan et al., (1998) Immunology 95: 427-436). Several cell types are able to effectively and efficiently mediate ADCC and include activating Fc receptor bearing NK cells, monocytes, dendritic cells and neutrophils. It is proposed that IL-2 will aid the immune reconstitution of severely lymphopenic Campath®-treated patients and augment ADCC via CD52 negative FcR-bearing immune effector cell types.

Campath-IH will be administered subcutaneously 2-3 times weekly. IL-2 (daily, weekly, twice weekly, thrice weekly) will be administered subcutaneously concomitant with or following Campath® administration.

### EXAMPLE 3

### Dosing Schedules of IL-2

Treatment with an IL-2 starts preferably on day 8 after the dose of 30 mg Alemtuzumab is reached. Subjects receive 3 weekly SC injections of IL-2 on the same days as Alemtuzumab for 11 weeks. The first six subjects receive IL-2 at a dose of 10 MIU SC three times weekly for total 11 weeks. After cessation of co-administration of Alemtuzumab and IL-2, a drug holiday will be given to the patient (four to six weeks). At this point the patient will receive cycles of IL-2 alone to restore T-cell counts. This regimen consists of administering an IL-2 twice a day for five consecutive days, followed by several weeks off of IL-2, for example, seven weeks off. (No IL-2 treatment is given during these weeks.) Each dose of IL-2 is 2-10 MIU, more usually 2-7.5 MIU, and an exemplary dose is 4.5 MIU, administered subcutaneously.

In case of dose-limiting toxicity (DLT) during co-administration of IL-2 and Alemtuzumab in two or more out of six patients, the dose of IL-2 will be reduced to 6 MIU three times weekly. If the dosing is tolerated without dose limiting toxicity, the dose will be increased in another six subjects to 14 MIU three times weekly.

If the dose of 14 MIU is tolerated well and no DLT is observed, more patients will be included at the same dose level up to a total number of 25. If 14 MIU three times weekly is not tolerated well during the co-administration, patients will be included at the previous dose level.

Safety will be evaluated based on CTC criteria, including clinical and laboratory changes. Efficacy will be evaluated based on the number of complete and partial responses (NCI Working Group, Cheson, B., et al., 1996) and on percent change of T cells and NK cells in the peripheral blood. T cell and NK cell and NK-T cell numbers will be correlated with IL-2 and IL-2R plasma levels. The pharmacokinetics of IL-2 will be evaluated in selected patients.

### EXAMPLE 4

### Calculation IL-2 Serum Concentration-Time Curves for Pharmaceutical Formulations of Aldesleukin and Variants Thereof

The area under the serum concentration-time curve (AUC) of Proleukin® IL-2 administered subcutaneously (SC) at 4.5 million international units (MIU) (equivalent to approximately 300/μg protein) was determined using data from an unpublished HIV study. Serum concentration time profiles were measured in 8 IL-2 naïve, HIV patients following an initial exposure to IL-2 dosing in this study. For each patient, the AUC was calculated using the linear trapezoidal rule up to the last measurable concentrations and extrapolated to 24 hours (Winnonlin software version 3.1, Pharsight Corporation, California). The average AUC₀₋₂₄, SD, and the lower and upper 95% confidence limits at 4.5 MIU dose are presented in Table 2.

The AUC₀₋₂₄ value of Proleukin® IL-2 administered SC at doses equivalent to 18 MIU (1200 μg) was estimated using data from three different studies where this IL-2 product was administered SC. Two are published studies, one in HIV patients (N=3) (Piscitelli et al. (1996) Pharmacotherapy 16(5):754-759) and one in cancer patients (N=7) (Kirchner et al. (1998) Br. J. Clin. Pharmacol. 46:5-10). The third is an unpublished study in which serum concentration time data were available from 6 cancer patients after SC doses of IL-2. The similarity of the AUC in cancer and HIV patients was previously established. The actual doses administered in these three studies ranged between 18 and 34 MIU. For the two published trials, the AUC up to 24 hours (AUC₀₋₂₄) values were normalized to 18 MIU dose by multiplying the AUC with the quotient of 18 and actual dose in MIU. For example, if the AUC₀₋₂₄ for a 20 MIU dose was calculated to be 400, the normalized AUC₀₋₂₄ would be 400·18/20=360. For the unpublished cancer-patient study, individual AUC values were calculated from the serum concentration time data using the linear trapezoidal rule up to the last measurable concentrations and extrapolated to 24 hours (Winnonlin software version 3.1, Pharsight Corporation, California) then were normalized to 18 MIU dose as noted above. The overall mean and SD for all three studies was calculated as the weighted average of the means and variances, respectively, using equations 1 and 2.

$$1. {X}_{p} = \sqrt{\frac{( {n}_{1} {X}_{1} + {n}_{2} {X}_{2} + {n}_{3} {X}_{3} )}{( {n}_{1} + {n}_{2} + {n}_{3} )}}$$

$$2. {X}_{p} = \sqrt{\frac{( {n}_{1} - 1 ) {s}_{1}^{2} + ( {n}_{2} - 1 ) {s}_{2}^{2} + ( {n}_{3} - 1 ) {s}_{3}^{2} )}{( {n}_{1} + {n}_{2} + {n}_{3} - 3 )}}$$

Where n₁, n₂, n₃, X₁, X₂, X₃ and s²₁, s²₂, s²₃; are the number of subjects, means, and variances for each of the three studies, respectively. Xₚ and SDₚ are estimates of the overall mean and standard deviation. The overall average AUC, SD, and the lower and upper 95% confidence limits at 18 MIU are also presented in Table 2.

TABLE 2
Average (±SD) AUC₀₋₂₄ obtained after initial
exposure to a single dose administration of
Proleukin ® IL-2 administered subcutaneously.

| Proleukin ® IL-2 | | | | |
|---|---|---|---|---|
| Dose | AUC₀₋₂₄ | | | |
| (MIU/μg) | (IU * hr/ml) | SD | LL of 95% CI¹ | UL of 95% CI¹ |
| 4.5/275 | 51 | 14 | 23 | 78 |
| 6.0/367² | 65 | | 22 | 107 |
| 7.5/458 | 79 | 29 | 21 | 137 |
| 18/1100 | 344 | 127 | 90 | 598 |

¹Upper (UL) and lower (LL) limits of the 95% confidence intervals (CI). 95% CI were calculated as the mean ± 2 SD.
²Values for 6.0 MIU are estimated based on actual values for 4.5 MIU and 7.5 MIU.

Similar to Proleukin® IL-2, L2-7001, a liquid formulation of monomeric IL-2, was administered to HIV patients at doses ranging from 50 to 180 μg (unpublished data). The exposures obtained from this study as measured by AUC are shown in Table 3. These exposure values were within the range of the exposure values generated using Proleukin® IL-2 (Table 1).

TABLE 3
Average (±SD) AUC₀₋₂₄ obtained after an initial
exposure to a single dose administration of the
monomeric IL-2 formulation L2-7001.

| L2-7001 Dose | AUC₀₋₂₄ | |
|---|---|---|
| (MIU/μg) | (IU * hr/ml) | SD |
| 0.82/50 | 65 | 11 |
| 1.5/90 | 110 | 36 |
| 2.2/135 | 143 | 41 |
| 2.9/180 | 275 | 99 |

The IL-2 exposure data (AUC) was obtained from the published literature where recombinant human native IL-2 was administered SC to 8 cancer patients at doses ranging from 0.1 MIU to 3.0 MIU. The reported average (% CV) AUCs for the 0.3, 1, and 3 MIU dose levels were 120 (38), 177 (36), and 359 (46) U*hr/ml (Gustavson (1998) J. Biol. Response Modifiers 1998:440-449). As indicated in Thompson et al. (1987) Cancer Research 47:4202-4207, the units measured in this study were normalized to BRMP units (Rossio et al. (1986) Lymphokine Research 5 (suppl 1):S13-S18), which was adopted later as international units (IU) by WHO (Gearing and Thorpe (1988) J. Immunological Methods 114:3-9). The AUC values generated under the study conditions also agree well with the established Proleukin® 5IL-2 exposure.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Those skilled in the art will recognize, or be able to ascertain using no more than routine experimentation, many equivalents to the specific embodiments of the invention described herein. Such equivalents are intended to be encompassed by the following claims.

The invention claimed is:

1. A method of treating chronic lymphocytic leukemia in a human subject, said method comprising administering to said subject at least one cycle of concurrent therapy with an anti-CD52 antibody and an interleukin-2 (IL-2).

2. The method of claim 1, wherein said IL-2 is recombinantly produced IL-2 having an amino acid sequence for human IL-2 or a variant thereof having at least 70% sequence identity to the amino acid sequence for human IL-2.

3. The method of claim 2, wherein said variant thereof is des-alanyl-1, serine 125 human interleukin-2.

4. The method of claim 1, wherein said anti-CD52 antibody is an immunologically active anti-CD52 antibody.

5. The method of claim 4, wherein said anti-CD52 antibody is Alemtuzumab or fragment thereof.

6. A method of treating chronic lymphocytic leukemia in a human subject, said method comprising administering to said subject at least one cycle of concurrent therapy with an anti-CD52 antibody and an interleukin-2 (IL-2), wherein said cycle comprises administering a therapeutically effective dose of an anti-CD52 antibody according to a weekly, twice-weekly, or thrice-weekly dosing schedule in combination with administration of a constant IL-2 dosing regimen, said constant IL-2 dosing regimen comprising administering a total weekly dose of an IL-2 to said subject.

7. The method of claim 6, wherein a first dose of an IL-2 is administered to said subject concurrently with a first dose of an anti-CD52 antibody.

8. The method of claim 7, wherein a first dose of an IL-2 is administered to said subject one week after a first dose of an anti-CD52 antibody is administered to said subject.

9. The method of claim 6, wherein said IL-2 is recombinantly produced IL-2 having an amino acid sequence for human IL-2 or a variant thereof having at least 70% sequence identity to the amino acid sequence for human IL-2.

10. The method of claim 9, wherein said variant thereof is des-alanyl-I, serine 125 human interleukin-2.

11. The method of claim 6, wherein said anti-CD52 antibody is an immunologically active anti-CD52 antibody.

12. The method of claim 11, wherein said anti-CD52 antibody is Alemtuzumab or fragment thereof.

13. The method of claim 6, wherein one or more subsequent cycles of concurrent therapy with IL-2 and anti-CD52 antibody is initiated about 1 month to about 6 months following completion of a first cycle or completion of any subsequent cycles of concurrent therapy with IL-2 and anti-CD52 antibody.

14. The method of claim 13, wherein T-cell counts are monitored in said subject to determine when each of said cycles is initiated, said cycles being initiated when T-cell count is less than 80% of the T-cell count at the conclusion of any previous cycle of concurrent therapy with an IL-2 and an anti-CD52 antibody.

15. The method of claim 6, wherein said total weekly dose of an IL-2 is in an amount that provides at least 50% of the NK stimulatory activity of a total weekly dose of Aldesleukin administered in a range of from about 1100 zig to about 1834 p. g.

16. A product containing an anti-CD52 antibody and an IL-2 as a combined preparation for simultaneous, separate, or sequential use in CLL therapy.

17. The product of claim 16, wherein said anti-CD52 antibody is an immunologically active anti-CD52 antibody.

18. The product of claim 16, wherein said anti-CD52 antibody is Alemtuzumab or fragment thereof.

19. The product of claim 16, wherein said anti-CD52 antibody is a human anti-CD52 antibody, a humanized anti-CD52 antibody, or a chimeric anti-CD52 antibody.

20. The product of claim 16, wherein said IL-2 is recombinantly produced IL-2 having an amino acid sequence for human IL-2 or a variant thereof having at least 70% sequence identity to the amino acid sequence for human IL-2.

21. The product of claim 20, wherein said variant thereof is des-alanyl-I, serine 125 human interleukin-2.

22. Use of an interleukin-2 (IL-2) in the preparation of a medicament for treating chronic lymphocytic leukemia (CLL) in a human subject previously administered with, or receiving administration of, an anti-CD52 antibody.

23. Use of an anti-CD52 antibody in the preparation of a medicament for treating CLL in a human subject previously administered with, or receiving administration of, an IL-2.

24. Use of an IL-2 in the preparation of a medicament for treating CLL in a human subject by separate, sequential or simultaneous administration with an anti-CD52 antibody.

25. Use of an anti-CD52 antibody in the preparation of a medicament for treating CLL in a human subject by separate, sequential or simultaneous administration with an IL-2.

26-32. (canceled)

33. A kit comprising an anti-CD52 antibody, an IL-2 and instructions for administering the IL-2, separately, simultaneously or sequentially with administration of the anti-CD52 antibody, to an individual suffering from CLL.

34. A kit according to claim 33 wherein the instructions are to administer the IL-2 following the administration of the anti-CD52 antibody.

35. A kit according to claim 34, wherein said IL-2 is recombinantly produced IL-2 having an amino acid sequence for human IL-2 or a variant thereof having at least 70% sequence identity to the amino acid sequence for human IL-2.

36. A kit according to claim 35, wherein said variant thereof is des-alanyl-1, serine 125 human interleukin-2.

37. A kit according to claim 36, wherein said anti-CD52 antibody is an immunologically active anti-CD52 antibody.

38. A kit according to claim 37, wherein said anti-CD52 antibody is Alemtuzumab or a fragment thereof.

39. A kit according to claim 37, wherein said anti-CD52 antibody is a human anti-CD52 antibody, a humanized anti-CD52 antibody, or a chimeric anti-CD52 antibody.
